SEQUENCE LISTING (8 sequences)

SEQ ID NO: 1 - DNA, 807 bp, Chlamydomonas rienhardtii
atggccgagtctgctggaaagcctctgaagcaccttgagtttgtgcacacctacgcgcacaagtttgcgagtggtgccgcttacgttgagggcggctaccagaaggccaagacctatgttcccgcggtcgctcagccctacatcgccaaagccgaggagacctgcctagcatacgccgctcctcttgcgacaaaggcgacggaccacgcagagaagattctccggagcaccgacgcacagctggacgcgctgtacgcggcctccgccagctggctgagcagctcccagaagctggccgactccaacatcgcggccttcaggggcgccgccgacaagtactacgacctggtcaagtccactgcgcagcacgtgacgtccaagctgcccaccgacctgtcggtggccaaggcccgcgagctgctgtcggcctcgctggagcaggccaaggctctggctgacccggacgctgcggtggcggcggcgctggatgcctggaccaagttcgcagccatcccggcggttgccaaggtgctgtccgccgcctccccgctgacgggcaagggcgtggcggccttcacggcggcacacgacctcctggtgcactcggcgctgtaccgctacggcgtgtcggtgggcgcctccaccctgggctgggccaccagcaccaccccctacaagttgagcgcggcttacctgtacccgctggtgcagcccgtggcggaccccgcgctggacaaggtgtccaagagcacctacgtcaacgcagccatcaagtactgggcgcccgcacccgtggcggccgcgtaa

SEQ ID NO: 2 - protein, 268 aa, Chlamydomonas rienhardtii
MAESAGKPLKHLEFVHTYAHKFASGAAYVEGGYQKAKTYVPAVAQPYIAKAEETCLAYAAPLATKATDHAEKILRSTDAQLDALYAASASWLSSSQKLADSNIAAFRGAADKYYDLVKSTAQHVTSKLPTDLSVAKARELLSASLEQAKALADPDAAVAAALDAWTKFAAIPAVAKVLSAASPLTGKGVAAFTAAHDLLVHSALYRYGVSVGASTLGWATSTTPYKLSAAYLYPLVQPVADPALDKVSKSTYVNAAIKYWAPAPVAAA

SEQ ID NO: 3 - DNA, 1553 bp, Volvox carteri
gataagaaagtagtcttgcatccatttgcggcatcgaagtattggcagctagcaaaccctttcactgaacttgtcgctatggctgacgaccgtaagctgaagcgcctgggctttgttgatgcctacacgcacaagcttgccaatggcgcagcttacgtcgagggtgcatacaagaaggtcaagccccttgtcccgcagcaagtgcagcccttcctcgcgaaggtggaggacgctgtgcttgcgtacacagctcctgtggttgccaaggcatccgaccaggcagagaagtttctgcgtattaccgacgagcaggttgattatctgtacgtggagacggctgcgtacctgacccagacgcgcaagttgacgcagagcaacatcgatactttccgctctgctgccgacaagtactaccagatggtcaagtccaccgcagactacttggcttcaaagctcagctatgacatctcggttcaggcggctcgcgatttcatcagcaagtctgtggagaaggccaaggagctttcggaccccgacgccgccgtgcgcatcgtttatgactcctggcagcaattcgcggccattccggcagttgccaagacgctggagaagaccgccccagtaaccaggaagggcttcgagaccttcatcgcggcgcatgatgcgctagtgagctcgcttgtgtacaagcgctccgtcagcctaggtgccaccacgctgggctgggccacaaccaccacgccctacaagctgggcgcgcagtatttgtaccccatggtccagtctgtggctgatccggcactggagaaggtggctaagagcacctacgtcaacgccactttgaagtactgggcgcctgtggcggcggcgtgaaaccctatggccattgttggattgccgtcatcttggggcaccaggccgttttctccttgtacgttgtataacatctgatccgtcctacgtattcttgctgtctaaacgatgggcgtgacctggggggttcatggccctgtgtcgcagcgaaaaaaaattgcggtcattgcggttgagtgttgggagatcttggtaccgaattatagttgtagttgcagttgttccccctgtgaattactgcgcgtacgacgtgatggataagaccgccaagcgaacacatttcatgatatatcgcgaggagggtggccagggttttcagtcccaaagccgttgatcgctggggtgaaggagagttgaagaagagggttttcggagagaatgaatgcttatttgtttttagtactgcaacaaaaatttagttgtaccgcatcttagttgtacggtaaaccgttgtgacattatcgaggttaattccccagtgcgcttgcagggtagtgttgtctttttggctgtttgttgctgcatgtacttttaggggtcgcaaagttatgtatttcttatatcttgtagtacagtttttgttgaaggccttgagggctgaaaagggcggaaaagatgacaaagatttagatggtaactatgtatgagacaactatttcctgtaaataccatggagaaac

SEQ ID NO: 4 - protein, 264 aa, Volvox carteri
MADDRKLKRLGFVDAYTHKLANGAAYVEGAYKKVKPLVPQQVQPFLAKVEDAVLAYTAPVVAKASDQAEKFLRITDEQVDYLYVETAAYLTQTRKLTQSNIDTFRSAADKYYQMVKSTADYLASKLSYDISVQAARDFISKSVEKAKELSDPDAAVRIVYDSWQQFAAIPAVAKTLEKTAPVTRKGFETFIAAHDALVSSLVYKRSVSLGATTLGWATTTTPYKLGAQYLYPMVQSVADPALEKVAKSTYVNATLKYWAPVAAA

SEQ ID NO: 5 - DNA, 828 bp, Haematococcus pluvialis
atgtcagagaagcagctgaagcgcttgggcttcgtgcatcagggagccagctatgcatacagctacacaggcacagccgagaaactgtacaagacagcgcgctccttcgccccaacctttgtggaacccaccttggcccaggttgaggatcgcgttgtggccatcacagccccagtggtggctcaggcgcaagacctcagcgagaaggcgttacacatcgccgatgaccaggtggactgcatcctgaacaccaccgacaaggcggtggcagacgggaagaagggcgtagttgattgcatgaacggcgtgaaggagatgcacgagaagaacatgcaaacctacatcgccacgagcaacagctactttgagtacatcaagggcatctccgactgggcaaaagataagctgaacccaattaagggcggccagcacgccctggacaccctgaacgccgcgattgccaaggctcaagaggcaactgaccccgacgtggcagctaagatggctctggatgcctggaacagctttgcatccgtgcctgtggtggccaaggtactggagacagccgacccagtcacgcagaccggcctgtcttccttctacaagctgcacgacaccctggtgagctggcccctgtacagcaaggtggtgtcaaccggggtgtccaccctgagctgggccacaaccaccatgccatacaagctgggcgcccagtacatgtaccccctggtgcagcccgtggctgacccagcattggccaagatcaccaacagcaaggtcatcaatggcacgctgtcgtactggaagccaactgcctcggcagcttga

SEQ ID NO: 6 - protein, 275 aa, Haematococcus pluvialis
MSEKQLKRLGFVHQGASYAYSYTGTAEKLYKTARSFAPTFVEPTLAQVEDRVVAITAPVVAQAQDLSEKALHIADDQVDCILNTTDKAVADGKKGVVDCMNGVKEMHEKNMQTYIATSNSYFEYIKGISDWAKDKLNPIKGGQHALDTLNAAIAKAQEATDPDVAAKMALDAWNSFASVPVVAKVLETADPVTQTGLSSFYKLHDTLVSWPLYSKVVSTGVSTLSWATTTMPYKLGAQYMYPLVQPVADPALAKITNSKVINGTLSYWKPTASAA

SEQ ID NO: 7 - DNA, 894 bp, Dunaliella parva
atggcgcccgcttccaagactgggccaaagggcagcaccactccccaggcccccacccccaacacagcaggccctcagctgcgacgcctggggtttgtgcgcagctacgcaggctctgccgcctccctgcttgcgcccatcctgctcacagtgcagagctttggtgaccgcgtgctggagagcgtgggcaccaaggagtcgctggccgcgtacaccacgccgctgctggagcgcactacggatatcggggacagcctgctcagcaccgtggaccagcaggtggactacatgctcaacacaggcaactcgatggtgcggaccaccagccagaccgtgactgacagcgtctcaggcgtgcgcgatatccacaactccaacctgtcctacctgagcaacgcgttccagacgctcctgaccaacctgcagcgcaccaccgactgggcgatcgagaacctgaaccccgtgcgtatcgcgcacactggctcagactgggccaaggcgacgttcgtgcgcgcacaggagctgctggatcccgacaccctgtacaacttcctgcaggagcgctgggctgcggtctcatccatccccgtagtgaaaggcatgctggacaccgcgcagcccatcacaaacgcatcctggagcgcgttcgtcggcctgcatgacttcctagtgaactcaagcctgtacaagttcagcgtggactctggcttctctgcatggggctgggctaagtcaaccaccccctacaagctgggcatgcagtacctgtaccccatcgtgcagcccgtcgcggaccctgcaatccacaaggtgagctcatccaaggtcgtgaacagcgtgctggactactgggcgcccacacctaatgcagcagccgcagctgcagcatcctga

SEQ ID NO: 8 - protein, 297 aa, Dunaliella parva
MAPASKTGPKGSTTPQAPTPNTAGPQLRRLGFVRSYAGSAASLLAPILLTVQSFGDRVLESVGTKESLAAYTTPLLERTTDIGDSLLSTVDQQVDYMLNTGNSMVRTTSQTVTDSVSGVRDIHNSNLSYLSNAFQTLLTNLQRTTDWAIENLNPVRIAHTGSDWAKATFVRAQELLDPDTLYNFLQERWAAVSSIPVVKGMLDTAQPITNASWSAFVGLHDFLVNSSLYKFSVDSGFSAWGWAKSTTPYKLGMQYLYPIVQPVADPAIHKVSSSKVVNSVLDYWAPTPNAAAAAAAS

(19) United States
(12) Patent Application Publication
(10) Pub. No.: US 2014/0274777 A1
(43) Pub. Date: Sep. 18, 2014

(54) LIPID DROPLET PROTEIN MARKERS FOR ALGAL OIL ACCUMULATION

(71) Applicant: Board of Trustees of Michigan State University, East Lansing, MI (US)

(72) Inventors: Christoph Benning, East Lansing, MI (US); Eric R. Moellering, East Lansing, MI (US); Chia-Hong Tsai, East Lansing, MI (US)

(73) Assignee: Board of Trustees of Michigan State University, East Lansing, MI (US)

(21) Appl. No.: 14/208,254

(22) Filed: Mar. 13, 2014

Related U.S. Application Data
(60) Provisional application No. 61/779,710, filed on Mar. 13, 2013.

(51) Int. Cl. G01N 33/566

(52) U.S. Cl. CPC G01N 33/566
USPC 506/9; 204/450; 435/6.11; 435/6.12; 435/7.1; 435/7.92; 435/287.2; 506/16; 506/18

(57) ABSTRACT

The application relates to apparatuses, methods and kits for detecting and quantifying oil content in algal samples.

This application claims benefit of the priority filing date of U.S. Provisional Patent Application No. 61/779,710, filed Mar. 13, 2013, the contents of which are specifically incorporated herein in their entirety.

### GOVERNMENT FUNDING

This invention was made with Government support under grant no. FA9550-11-1-0264 awarded by the Air Force Office of Scientific Research. The Government has certain rights in the invention.

### BACKGROUND

Oils (triacylglycerols) from algae are a promising feedstock for renewable transportation energy. The process of large scale aquaculture for algal biofuel production is in its infancy, and tools that allow monitoring of oil accumulation in algae are not readily available.

### SUMMARY

As described herein, a lipid droplet associated protein, Major Lipid Droplet Protein (MLDP), has been identified in the green algae Chlamydomonas reinhardtii and can be used to detect and/or quantify oil content in algal tissues. Although MLDP is not directly involved in oil synthesis it is surprisingly effective as a measure of oil accumulation. Methods, kits, and tools are described herein that are useful for detecting and quantifying oil accumulation in algae by detection and/or quantification of MLDP. The methods have several advantages. For example, the methods simplify detection, monitoring and quantifying oil formation in algae (e.g., in algal production ponds), so that less effort, time, resources/instrumentation, and training are needed compared to direct quantification of oils. The methods described herein can also be employed to detect potential cross-contamination of different algal species (e.g., different green algal strains or species) so that the types of oil-producing algae can be adjusted to optimize oil production.

### DESCRIPTION OF THE FIGURES

FIG. 1 illustrates that MLDP antiserum is specific for a single 28 kDa band from nitrogen depleted Chlamydomonas reinhardtii. A 1:1,000 dilution prebleed was used as a negative control (lanes 1 and 2). The specificity of the antiserum is shown by blots exposed to 1:1,000 (lanes 3 and 4) and 1:10,000 (lanes 4 and 5) diluted testbleeds. The electrophoretically separated proteins were from cells grown in the presence of nitrogen (see lanes 1, 3, and 5) and in cells grown in the absence of nitrogen (see lanes 2, 4, and 6).

FIG. 2A-2C illustrates that MLDP abundance parallels the amount of triacylglycerol. Cells were grown in nitrogen depleted media (−N, for 0 to 60 hours), followed by nitrogen resupply (+N, for 0 to 36 hours). FIG. 2A graphically illustrates MLDP abundance detected by immunoblot. MLDP is indicated with the arrow. FIG. 2B graphically illustrates the results of gas chromatography with flame ionization detector analyses of triacylglycerol fatty acids relative to total fatty acids. FIG. 2C is a bar graph of the pixels from an MLDP immunoblot that were quantified using the National Institutes of Health (NIH) ImageJ image processing software. The Y-axis indicates the absolute number of pixel detected.

FIG. 3A-3D illustrates the distribution of MLDP at the surface of lipid droplets. Lipid droplet formation was induced by nitrogen depletion (24 hours). FIG. 3A shows images of cells recorded by conventional thin-section electron microscopy. FIG. 3B-3D show images of immunogold labeled cells showing MLDP expression. Arrows indicate the label (15 nm gold) location at the surface of the lipid droplet. LD, lipid droplet; N, nucleus.

FIG. 4A-4B show that MLDP can recruit different proteins during nitrogen depletion and nitrogen recovery. FIG. 4A shows an immunoblot of MLDP from lipid droplets that were isolated from nitrogen depleted cells and solubilized with 1% dodecylmaltoside, then separated with 4-16% high definition native polyacrylamide gel electrophoresis (HDN-PAGE) in the first dimension (left to right) and 4-20% sodium dodecyl sulfate polyacrylamide gel electrophoresis in the second dimension (top to bottom) followed by immunoblot with anti-MLDP antibody. FIG. 4B shows an immunoblot of MLDP from lipid droplets from nitrogen depleted and nitrogen-restored cells were separated by one-dimensional high definition native polyacrylamide gel electrophoresis.

### DETAILED DESCRIPTION

Methods, apparatus, and kits for detecting oil accumulation in algae are described herein. Such methods, apparatus, or kits can be used to detect, monitor and/or quantify oil formation in a variety of algal species and strains by detection of the lipid droplet specific protein—the Major Lipid Droplet Protein (MLDP). While MLDP expression is directly correlated with oil content, MLDP is not directly involved in the synthesis or degradation of oil. Instead, MLDP is a protein present in oil droplets.

### Lipid Droplets

Eukaryotic cells store oils in the chemical form of triacylglycerols in distinct organelles, often called lipid droplets. These dynamic storage compartments have been studied in the context of human health. However, the inventors are interested in algae as a source of vegetable oils for human consumption and for chemical or biofuel feedstocks. Many algae accumulate oils, particularly under conditions limiting to growth. While algae could be sustainable feedstock for biofuel production, little is currently known at the cellular or molecular levels with regard to oil accumulation in algae. Nor are the structural proteins and enzymes involved in the biogenesis, maintenance, and degradation of algal oil storage compartments well understood.

As described herein, the accumulation of triacylglycerols and the formation of lipid droplets during nitrogen deprivation were investigated in the model green alga Chlamydomonas reinhardtii. Mass spectrometry identified 259 proteins in a lipid droplet-enriched fraction, among them a major protein, tentatively designated major lipid droplet protein (MLDP). This protein is specific to the green algal lineage of photosynthetic organisms. Repression of MLDP gene expression using an RNA interference approach led to increased lipid droplet size, but no change in triacylglycerol content or metabolism was observed.

Major Lipid Droplet Protein (MLDP)

The Major Lipid Droplet Protein (MLDP) is a perilipin-type protein present in photosynthetic organisms. The lipid droplet-enriched fraction of the green algal photosynthetic organism Chlamydomonas reinhardtii was investigated. Lipid droplet associated proteins from C. reinhardtii were analyzed based on Coomassie Brilliant Blue staining and mass spectrometric analysis of respective gel slices. The new structural protein, MLDP, was characterized through proteomics research (Moellering & Benning, Eukaryot Cell, 9:97 (2010)).

Preliminary experiments indicated that MLDP could be a functional analogue of perilipin. RNA silencing of MLDP transcripts led to increased size of lipid droplets without affecting the total triacylglycerol content or metabolism. The increase in lipid droplet size may compensate for the need of the structural proteins by reducing the overall surface area. In addition to a having a structural role, MLDP was shown to recruit other proteins such as lipases or regulatory proteins to lipid droplets and thereby indirectly influence lipogenesis and lipolysis. Because MLDP affects lipid droplet dynamics and size, assays detecting or quantifying lipid droplet associated proteins, such as MLDP, provides a robust tool to track oil accumulation in various organisms and production levels in cultures.

As described herein, the MLDP protein and its transcript abundance is induced under conditions which oils accumulate in Chlamydomonas rienhardtii.

The MLDP nucleotide sequence is as follows (SEQ ID NO: 1):

| | |
|---|---|
| 1 | ATGGCCGAGT CTGCTGGAAA GCCTCTGAAG CACCTTGAGT |
| 41 | TTGTGCACAC CTACGCGCAC AAGTTTGCGA GTGGTGCCGC |
| 81 | TTACGTTGAG GGCGGCTACC AGAAGGCCAA GACCTATGTT |
| 121 | CCCGCGGTCG CTCAGCCCTA CATCGCCAAA GCCGAGGAGA |
| 161 | CCTGCCTAGC ATACGCCGCT CCTCTTGCGA CAAAGGCGAC |
| 201 | GGACCACGCA GAGAAGATTC TCCGGAGCAC CGACGCACAG |
| 241 | CTGGACGCGC TGTACGCGGC CTCCGCCAGC TGGCTGAGCA |
| 281 | GCTCCCAGAA GCTGGCCGAC TCCAACATCG CGGCCTTCAG |
| 321 | GGGCGCCGCC GACAAGTACT ACGACCTGGT CAAGTCCACT |
| 361 | GCGCAGCACG TGACGTCCAA GCTGCCCACC GACCTGTCGG |
| 401 | TGGCCAAGGC CCGCGAGCTG CTGTCGGCCT CGCTGGAGCA |
| 441 | GGCCAAGGCT CTGGCTGACC CGGACGCTGC GGTGGCGGCG |
| 481 | GCGCTGGATG CCTGGACCAA GTTCGCAGCC ATCCCGGCGG |
| 521 | TTGCCAAGGT GCTGTCCGCC GACCCCGCGC TGACGGGCAA |
| 561 | GGGCGTGGCG GCCTTCACGG CGGCACACGA CCTCCTGGTG |
| 601 | CACTCGGCGC TGTACCGCTA CGGCGTGTCG GTGGGCGCCT |
| 641 | CCACCCTGGG CTGGGCCACC AGCACCACCC CCTACAAGTT |
| 681 | GAGCGCGGCT TACCTGTACC CGCTGGTGCA GCCCGTGGCG |
| 721 | GACCCCGCGC TGGACAAGGT GTCCAAGAGC ACCTACGTCA |
| 761 | ACGCAGCCAT CAAGTACTGG GCGCCCGCAC CCGTGGCGGC |
| 801 | CGCGTAA |

The MLDP amino acid sequence is as follows (SEQ ID NO: 2):

| | |
|---|---|
| 1 | MAESAGKPLK HLEFVHTYAH KFASGAAYVE GGYQKAKTYV |
| 41 | PAVAQPYIAK AEETCLAYAA PLATKATDHA EKILRSTDAQ |
| 81 | LDALYAASAS WLSSSQKLAD SNIAAFRGAA DKYYDLVKST |
| 121 | AQHVTSKLPT DLSVAKAREL LSASLEQAKA LADPDAAVAA |
| 161 | ALDAWTKFAA IPAVAKVLSA ASPLTGKGVA AFTAAHDLLV |
| 201 | HSALYRYGVS VGASTLGWAT STTPYKLSAA YLYPLVQPVA |
| 241 | DPALDKVSKS TYVNAAIKYW APAPVAAA |

Homologues of MLDP are present in other algal species. For example, a nucleic acid from Volvox carteri has about 68% sequence identity to the Chlamydomonas rienhardtii nucleic acid with SEQ ID NO.1 (see accession number XM_{—}002958607.1, GI:302854287 in the database at ncbi.nlm.nih.gov/nucleotide/302854287?report=genbank&log$=nuclalign&blast_rank=2&RID=KHTXDYPE016). This Volvox carteri nucleic acid has the following sequence (SEQ ID NO:3)

| | |
|---|---|
| 1 | GATAAGAAAG TAGTCTTGCA TCCATTTGCG GCATCGAAGT |
| 41 | ATTGGCAGCT AGCAAACCCT TTCACTGAAC TTGTCGCTAT |
| 61 | GGCTGACGAC CGTAAGCTGA AGCGCCTGGG CTTTGTTGAT |
| 121 | GCCTACACGC ACAAGCTTGC CAATGGCGCA GCTTACGTCG |
| 161 | AGGGTGCATA CAAGAAGGTC AAGCCCCTTG TCCCGCAGCA |
| 201 | AGTGCAGCCC TTCCTCGCGA AGGTGGAGGA CGCTGTGCTT |
| 241 | GCGTACACAG CTCCTGTGGT TGCCAAGGCA TCCGACCAGG |
| 281 | CAGAGAAGTT TCTGCGTATT ACCGACGAGC AGGTTGATTA |
| 321 | TCTGTACGTG GAGACGGCTG CGTACCTGAC CCAGACGCGC |
| 361 | AAGTTGACGC AGAGCAACAT CGATACTTTC CGCTCTGCTG |
| 401 | CCGACAAGTA CTACCAGATG GTCAAGTCCA CCGCAGACTA |
| 461 | CTTGGCTTCA AAGCTCAGCT ATGACATCTC GGTTCAGGCG |
| 481 | GCTCGCGATT TCATCAGCAA GTCTGTGGAG AAGGCCAAGG |
| 521 | AGCTTTCGGA CCCCGACGCC GCCGTGCGCA TCGTTTATGA |
| 561 | CTCCTGGCAG CAATTCGCGG CCATTCCGGC AGTTGCCAAG |
| 601 | ACGCTGGAGA AGACCGCCCC AGTAACCAGG AAGGGCTTCG |
| 641 | AGACCTTCAT CGCGGCGCAT GATGCGCTAG TGAGCTCGCT |
| 681 | TGTGTACAAG CGCTCCGTCA GCCTAGGTGC CACCACGCTG |
| 721 | GGCTGGGCCA CAACCACCAC GCCCTACAAG CTGGGCGCGC |
| 761 | AGTATTTGTA CCCCATGGTC CAGTCTGTGG CTGATCCGGC |
| 801 | ACTGGAGAAG GTGGCTAAGA GCACCTACGT CAACGCCACT |
| 841 | TTGAAGTACT GGGCGCCTGT GGCGGCGGCG TGAAACCCTA |
| 881 | TGGCCATTGT TGGATTGCCG TCATCTTGGG GCACCAGGCC |
| 921 | GTTTTCTCCT TGTACGTTGT ATAACATCTG ATCCGTCCTA |
| 961 | CGTATTCTTG CTGTCTAAAC GATGGGCGTG ACCTGGGGGG |
| 1001 | TTCATGGCCC TGTGTCGCAG CGAAAAAAAA TTGCGGTCAT |
| 1041 | TGCGGTTGAG TGTTGGGAGA TCTTGGTACC GAATTATAGT |
| 1081 | TGTAGTTGCA GTTGTTCCCC CTGTGAATTA CTGCGCGTAC |
| 1121 | GACGTGATGG ATAAGACCGC CAAGCGAACA CATTTCATGA |
| 1161 | TATATCGCGA GGAGGGTGGC CAGGGTTTTC AGTCCCAAAG |
| 1201 | CCGTTGATCG CTGGGGTGAA GGAGAGTTGA AGAAGAGGGT |
| 1241 | TTTCGGAGAG AATGAATGCT TATTTGTTTT TAGTACTGCA |
| 1281 | ACAAAAATTT AGTTGTACCG CATCTTAGTT GTACGGTAAA |
| 1321 | CCGTTGTGAC ATTATCGAGG TTAATTCCCC AGTGCGCTTG |
| 1361 | CAGGGTAGTG TTGTCTTTTT GGCTGTTTGT TGCTGCATGT |
| 1401 | ACTTTTAGGG GTCGCAAAGT TATGTATTTC TTATATCTTG |
| 1441 | TAGTACAGTT TTTGTTGAAG GCCTTGAGGG CTGAAAAGGG |
| 1481 | CGGAAAAGAT GACAAAGATT TAGATGGTAA CTATGTATGA |
| 1521 | GACAACTATT TCCTGTAAAT ACCATGGAGA AAC |

The SEQ ID NO: 3 nucleic acid encodes a protein with NCBI accession number XP_{—}002958653.1 (GI:302854288) without an apparent function, provided below as SEQ ID NO:4.

| | |
|---|---|
| 1 | MADDRKLKRL GFVDAYTHKL ANGAAYVEGA YKKVKPLVPQ |
| 41 | QVQPFLAKVE DAVLAYTAPV VAKASDQAEK FLRITDEQVD |
| 81 | YLYVETAAYL TQTRKLTQSN IDTFRSAADK YYQMVKSTAD |
| 121 | YLASKLSYDI SVQAARDFIS KSVEKAKELS DPDAAVRIVY |
| 161 | DSWQQFAAIP AVAKTLEKTA PVTRKGFETF IAAHDALVSS |
| 201 | LVYKRSVSLG ATTLGWATTT TPYKLGAQYL YPMVQSVADP |
| 241 | ALEKVAKSTY VNATLKYWAP VAAA |

Haematococcus pluvialis is another species of algae with a nucleic acid sequence having 71% sequence identity to SEQ ID NO:1. This Haematococcus pluvialis nucleic acid has NCBI accession number HQ213938.1 (GI:307829118) and is provided below as SEQ ID NO:5.

| | |
|---|---|
| 1 | ATGTCAGAGA AGCAGCTGAA GCGCTTGGGC TTCGTGCATC |
| 41 | AGGGAGCCAG CTATGCATAC AGCTACACAG GCACAGCCGA |
| 81 | GAAACTGTAC AAGACAGCGC GCTCCTTCGC CCCAACCTTT |
| 121 | GTGGAACCCA CCTTGGCCCA GGTTGAGGAT CGCGTTGTGG |
| 161 | CCATCACAGC CCCAGTGGTG GCTCAGGCGC AAGACCTCAG |
| 201 | CGAGAAGGCG TTACACATCG CCGATGACCA GGTGGACTGC |
| 241 | ATCCTGAACA CCACCGACAA GGCGGTGGCA GACGGGAAGA |
| 281 | AGGGCGTAGT TGATTGCATG AACGGCGTGA AGGAGATGCA |
| 321 | CGAGAAGAAC ATGCAAACCT ACATCGCCAC GAGCAACAGC |
| 361 | TACTTTGAGT ACATCAAGGG CATCTCCGAC TGGGCAAAAG |
| 401 | ATAAGCTGAA CCCAATTAAG GGCGGCCAGC ACGCCCTGGA |
| 441 | CACCCTGAAC GCCGCGATTG CCAAGGCTCA AGAGGCAACT |
| 481 | GACCCCGACG TGGCAGCTAA GATGGCTCTG GATGCCTGGA |
| 521 | ACAGCTTTGC ATCCGTGCCT GTGGTGGCCA AGGTACTGGA |
| 561 | GACAGCCGAC CCAGTCACGC AGACCGGCCT GTCTTCCTTC |
| 601 | TACAAGCTGC ACGACACCCT GGTGAGCTGG CCCCTGTACA |
| 641 | GCAAGGTGGT GTCAACCGGG GTGTCCACCC TGAGCTGGGC |
| 681 | CACAACCACC ATGCCATACA AGCTGGGCGC CCAGTACATG |
| 721 | TACCCCCTGG TGCAGCCCGT GGCTGACCCA GCATTGGCCA |
| 761 | AGATCACCAA CAGCAAGGTC ATCAATGGCA CGCTGTCGTA |
| 801 | CTGGAAGCCA ACTGCCTCGG CAGCTTGA |

The protein encoded by the SEQ ID NO:5 nucleic has NCBI accession number ADN95182.1 (GI:307829119) and is provided below as SEQ ID NO:6.

| | |
|---|---|
| 1 | MSEKQLKRLG FVHQGASYAY SYTGTAEKLY KTARSFAPTF |
| 41 | VEPTLAQVED RVVAITAPVV AQAQDLSEKA LHIADDQVDC |
| 81 | ILNTTDKAVA DGKKGVVDCM NGVKEMHEKN MQTYIATSNS |
| 121 | YFEYIKGISD WAKDKLNPIK GGQHALDTLN AAIAKAQEAT |
| 161 | DPDVAAKMAL DAWNSFASVP VVAKVLETAD PVTQTGLSSF |
| 201 | YKLHDTLVSW PLYSKVVSTG VSTLSWATTT MPYKLGAQYM |
| 241 | YPLVQPVADP ALAKITNSKV INGTLSYWKP TASAA |

Dunaliella parva is another species of algae with a nucleic acid sequence having a segment with 77% sequence identity to SEQ ID NO:1. This Dunaliella parva nucleic acid has NCBI accession number JQ011392.1 (GI:363901034) and is provided below as SEQ ID NO:7.

| | |
|---|---|
| 1 | ATGGCGCCCG CTTCCAAGAC TGGGCCAAAG GGCAGCACCA |
| 41 | CTCCCCAGGC CCCCACCCCC AACACAGCAG GCCCTCAGCT |
| 61 | GCGACGCCTG GGGTTTGTGC GCAGCTACGC AGGCTCTGCC |
| 121 | GCCTCCCTGC TTGCGCCCAT CCTGCTCACA GTGCAGAGCT |
| 161 | TTGGTGACCG CGTGCTGGAG AGCGTGGGCA CCAAGGAGTC |
| 201 | GCTGGCCGCG TACACCACGC CGCTGCTGGA GCGCACTACG |
| 241 | GATATCGGGG ACAGCCTGCT CAGCACCGTG GACCAGCAGG |
| 281 | TGGACTACAT GCTCAACACA GGCAACTCGA TGGTGCGGAC |
| 321 | CACCAGCCAG ACCGTGACTG ACAGCGTCTC AGGCGTGCGC |
| 361 | GATATCCACA ACTCCAACCT GTCCTACCTG AGCAACGCGT |
| 401 | TCCAGACGCT CCTGACCAAC CTGCAGCGCA CCACCGACTG |
| 441 | GGCGATCGAG AACCTGAACC CCGTGCGTAT CGCGCACACT |
| 481 | GGCTCAGACT GGGCCAAGGC GACGTTCGTG CGCGCACAGG |
| 521 | AGCTGCTGGA TCCCGACACC CTGTACAACT TCCTGCAGGA |
| 561 | GCGCTGGGCT GCGGTCTCAT CCATCCCCGT AGTGAAAGGC |
| 601 | ATGCTGGACA CCGCGCAGCC CATCACAAAC GCATCCTGGA |
| 641 | GCGCGTTCGT CGGCCTGCAT GACTTCCTAG TGAACTCAAG |
| 681 | CCTGTACAAG TTCAGCGTGG ACTCTGGCTT CTCTGCATGG |
| 721 | GGCTGGGCTA AGTCAACCAC CCCCTACAAG CTGGGCATGC |
| 761 | AGTACCTGTA CCCCATCGTG CAGCCCGTCG CGGACCCTGC |
| 801 | AACACAGCAG GTGAGCTCAT CCAAGGTCGT GACCAGCAGG |
| 841 | CTGGACTACT GGGCGCCCAC ACCTAATGCA GCAGCCGCAG |
| 881 | CTGCAGCATC CTGA |

The protein encoded by the SEQ ID NO:7 nucleic has NCBI accession number AEW43286.1 (GI:363901035) and is provided below as SEQ ID NO:8.

| | |
|---|---|
| 1 | MAPASKTGPK GSTTPQAPTP NTAGPQLRRL GFVRSYAGSA |
| 41 | ASLLAPILLT VQSFGDRVLE SVGTKESLAA YTTPLLERTT |
| 81 | DIGDSLLSTV DQQVDYMLNT GNSMVRTTSQ TVTDSVSGVR |
| 121 | DIHNSNLSYL SNAFQTLLTN LQRTTDWAIE NLNPVRIAHT |
| 161 | GSDWAKATFV RAQELLDPDT LYNFLQERWA AVSSIPVVKG |
| 201 | MLDTAQPITN ASWSAFVGLH DFLVNSSLYK FSVDSGFSAW |
| 241 | GWAKSTTPYK LGMQYLYPIV QPVADPAIHK VSSSKVVNSV |
| 281 | LDYWAPTPNA AAAAAAS |

Any of these nucleic acids, proteins and fragments thereof can be detected and/or quantified to detect and quantify the oil content of samples of algae.

### Methods

Methods are described herein for detecting, monitoring and quantifying oil content in algae. Such methods involve detection of either an MLDP nucleic acid or a polypeptide. As illustrated herein, the accumulation of MLDP directly correlates with the accumulation of oil in algae. For example, when MLDP levels or expression increase by 10%, the percentage of triacylglycerol fatty acids relative to total fatty acids increases by about 7-15%. When MLDP levels or expression increase by about 2%, the percentage of triacylglycerol fatty acids relative to total fatty acids increases by about 1.5-2.5%. When MLDP levels or expression increase ten-fold, the percentage of triacylglycerol fatty acids relative to total fatty acids increases by about 10 to about 20 fold. Hence, MLDP is a sensitive and quantitatively accurate indicator of oil content in algae.

A method of detecting oil content in a sample of algae is described herein that includes detecting MLDP in the sample. Such a method can also include quantifying MLDP expression in the sample. The quantity of MLDP directly correlates with the oil content of algae in the sample.

For example, the sample can be an algae sample such as an algae maintained or grown in culture, or in the field (e.g., in a pond). Different types of algae can be evaluated pursuant to the methods described herein. For example, the algae can be Chlamydomonas, Chlorophyta (green algae), Rhodophyta (red algae), or Phaeophyceae (brown algae).

Moreover, the algae evaluated can have a mutation in a gene encoding a polypeptide associated with lipid droplets. The sample can contain recombinant algae. For example, the sample can be sample of recombinant red, green or brown alga. Such recombinant algal cells can have a genome with a heterologous expression cassette that encodes a polypeptide of interest (e.g., a polypeptide involved in oil production or storage).

MLDP and related gene expression levels can be detected, monitored and/or quantified using any available procedure. For example, MLDP expression levels can be detected, monitored and/or quantified by detecting MLDP mRNA levels or MLDP protein levels. Expression levels of mRNA with sequences similar to MLDP can also be detected, monitored and/or quantified to evaluate oil content of algae. Similarly, proteins with functions and/or structures similar to the MLDP proteins can also be detected, monitored and/or quantified to assess oil content in algae.

The SEQ ID NO:1, 3, 5, and 7 sequences are provided herein as examples of MLDP and MLDP-related nucleic acids. However, nucleic acids with similar sequences such as those with at least about 65% sequence identity, or at least about 70% sequence identity, or at least about 75% sequence identity, or at least about 80% sequence identity, or at least about 85% sequence identity, or at least about 90% sequence identity, or at least about 95% sequence identity to any of SEQ ID NO:1, 3, 5, and 7 can be detected, monitored and/or quantified to assess oil content in algae.

The SEQ ID NO:2, 4, 6, and 8 sequences are provided herein as examples of MLDP and MLDP-related proteins. However, proteins with similar sequences such as those with at least about 65% sequence identity, or at least about 70% sequence identity, or at least about 75% sequence identity, or at least about 80% sequence identity, or at least about 85% sequence identity, or at least about 90% sequence identity, or at least about 95% sequence identity to any of SEQ ID NO:2, 4, 6, and 8 can be detected, monitored and/or quantified to assess oil content in algae.

### Detection of RNA Expression

Expression of MLDP mRNA and related RNAs can be assessed using any available technique for detecting and/or quantifying nucleic acids. Non-limiting examples of such techniques include microarray analysis, Northern blotting, nuclease protection assays, RNA fingerprinting, polymerase chain reaction, ligase chain reaction, Qbeta replicase, isothermal amplification method, strand displacement amplification, transcription based amplification systems, quantitative nucleic acid amplification assays (e.g., polymerase chain reaction assays), combined reverse transcription/nucleic acid amplification, nuclease protection (SI nuclease or RNAse protection assays), Serial Analysis Gene Expression (SAGE), next generation sequencing, gene expression microarray, as well as other methods.

A standard Northern blot assay can be used to ascertain an RNA transcript size, and the relative amounts of mRNA in a sample, in accordance with conventional Northern hybridization techniques known to those persons of ordinary skill in the art. In Northern blots, RNA samples are first separated by size via electrophoresis in an agarose gel under denaturing conditions. The RNA is then transferred to a membrane, cross-linked and hybridized with a labeled probe (e.g., a probe linked to a reporter molecule). Nonisotopic or high specific activity radiolabeled probes can be used including random-primed, nick-translated, or PCR-generated DNA probes, in vitro transcribed RNA probes, and oligonucleotides. Additionally, sequences with only partial homology (e.g., MLDP probes selected from cDNA or genomic DNA fragments that might contain an exon from a different species) may be used as probes. The labeled probe can be a labeled cDNA; a full-length, single stranded labeled RNA or DNA, or a labeled fragment of that RNA or DNA sequence. The label employed can be any available reporter molecule, for example, any of the reporter molecules described herein.

Such a RNA or DNA (or fragments thereof) may serve as a probe, for example, when it is at least 9, at least 10, at least 11, at least 12, at least 13, at least 14, at least 15, or at least 16 consecutive nucleotides in length. In some embodiments, the probe is about 9, about 10, about 11, about 12, about 13, about 14, about 15, about 16, about 17, about 18, about 19, about 20, about 21 or about 22 consecutive nucleotides in length. In further embodiments, the probe may be at least 20, at least 30, at least 50, or at least 70 consecutive nucleotides in length. The primers and/or probes can be less than about 80, less than about 70, less than about 60, less than about 50, less than about 45, less than about 40, less than about 39, less than about 38, less than about 37, less than about 36, less than about 35, less than about 34, less than about 33, less than about 32, less than about 31, or less than about 30 consecutive nucleotides in length.

The probe can be labeled by linkage to a reporter molecule using any of the many different methods available to those skilled in this art. The reporter molecules most commonly employed for these studies are radioactive elements, enzymes, chemicals that fluoresce when exposed to ultraviolet light, and others. A number of fluorescent materials are known and can be utilized as reporter molecules. These include, but are not limited to, fluorescein, rhodamine, auramine, Texas Red, AMCA blue and Lucifer Yellow. The radioactive reporter molecules can be detected by any of the currently available counting procedures. Non-limiting examples of isotopes include ³H, ¹⁴C, ³²P, ³⁵S, ³⁶Ci, ⁵¹Cr, ⁵⁷Co, ⁵⁸Co, ⁵⁹Fe, ⁹⁰Y, ¹²⁵I, ¹³¹I, and ¹⁸⁶Re.

Enzyme reporter molecules are likewise useful, and can be detected by any available colorimetric, spectrophotometric, fluorospectrophotometric, amperometric or gasometric technique. The enzyme can conjugated to a probe, or to a secondary molecule that binds to hybrid formed by the probe and an MLDP mRNA, by reaction with bridging molecules such as carbodiimides, diisocyanates, glutaraldehyde and the like. Any enzymes known to one of skill in the art can be utilized. Examples of such enzymes include, but are not limited to, peroxidase, beta-D-galactosidase, urease, glucose oxidase plus peroxidase and alkaline phosphatase. U.S. Pat. Nos. 3,654,090, 3,850,752, and 4,016,043 are referred to by way of example for their disclosure of alternate labeling material and methods.

Nuclease protection assays such as ribonuclease protection assays and Si nuclease assays can be used to detect and quantify specific mRNAs. In nuclease protection assays, an antisense probe (labeled with, e.g., radiolabeled or nonisotopic reporter molecules) hybridizes in solution to an RNA sample. Following hybridization, single-stranded, unhybridized probe and RNA are degraded by nucleases. An acrylamide gel is used to separate the remaining protected fragments. Typically, solution hybridization is more efficient than membrane-based hybridization, and it can accommodate up to 100 μg of sample RNA, compared with the 20-30 μg maximum of blot hybridizations.

A ribonuclease protection assay typically employs RNA probes. Oligonucleotides and other single-stranded DNA probes can be used in assays containing S1 nuclease. The single-stranded, antisense probe is ideally completely homologous to target RNA to prevent cleavage of the probe:target hybrid by nuclease.

Serial Analysis Gene Expression (SAGE), which is described in e.g., Velculescu et al., 1995, Science 270:484-7; Carulli, et al., 1998, Journal of Cellular Biochemistry Supplements 30/31:286-96, can also be used to determine RNA abundances in samples.

Quantitative reverse transcriptase PCR (qRT-PCR) can also be used to determine the expression levels of mRNA (see, e.g., U.S. Patent Application Publication No. 2005/0048542A1). The first step in gene expression profiling by RT-PCR is the reverse transcription of the RNA template into cDNA, followed by its exponential amplification in a PCR reaction. The two most commonly used reverse transcriptases are avilo myeloblastosis virus reverse transcriptase (AMV-RT) and Moloney murine leukemia virus reverse transcriptase (MLV-RT). The reverse transcription step is typically primed using specific primers, random hexamers, or oligo-dT primers, depending on the circumstances and the goal of expression profiling. For example, extracted RNA can be reverse-transcribed using a GeneAmp RNA PCR kit (Perkin Elmer, Calif., USA), following the manufacturer's instructions. The derived cDNA can then be used as a template in the subsequent PCR reaction.

Although the PCR step can use a variety of thermostable DNA-dependent DNA polymerases, commonly employed polymerases include the Taq DNA polymerase, which has a 5′-3′ nuclease activity but lacks a 3′-5′ proofreading endonuclease activity. TagMan® PCR typically utilizes the 5′-nuclease activity of Taq or Tth polymerase to hydrolyze a hybridization probe bound to its target amplicon, but any enzyme with similar or equivalent 5′ nuclease activity can be used. Two oligonucleotide primers are used to generate an amplicon typical of a PCR reaction. A third oligonucleotide, or probe, is designed to detect nucleotide sequence located between the two PCR primers. The probe is non-extendible by Taq DNA polymerase enzyme, and is labeled with a reporter fluorescent dye and a quencher fluorescent dye. Any laser-induced emission from the reporter dye is quenched by the quenching dye when the two dyes are located close together as they are on the probe. During the amplification reaction, the Taq DNA polymerase enzyme cleaves the probe in a template-dependent manner. The resultant probe fragments disassociate in solution, and signal from the released reporter dye is free from the quenching effect of the second fluorophore. One molecule of reporter dye is liberated for each new molecule synthesized, and detection of the unquenched reporter dye provides the basis for quantitative interpretation of the data.

TagMan® RT-PCR can be performed using commercially available equipment, such as, for example, ABI PRISM 7700™. Sequence Detection System™ (Perkin-Elmer-Applied Biosystems, Foster City, Calif., USA), or Lightcycler (Roche Molecular Biochemicals, Mannheim, Germany). In one embodiment, the 5′ nuclease procedure is run on a real-time quantitative PCR device such as the ABI PRISM 7700™ Sequence Detection System™. The system consists of a thermocycler, laser, charge-coupled device (CCD), camera and computer. The system includes software for running the instrument and for analyzing the data.

The detected quantity of Major Lipid Droplet Protein mRNA or DNA can be compared to a control (known) amount or concentration of Major Lipid Droplet Protein (MLDP) mRNA or DNA.

### Detection of Protein

MLDP and related proteins can be detected and/or quantified using any technique available to one of skill in the art. Examples of such methods include Western blotting, enzyme-linked immunosorbent assays (ELISAs), radioimmunoassay, immunocyto-chemistry, immunohistochemistry, flow cytometry, immunoprecipitation, one- and two-dimensional electrophoresis, immunoPCR (iPCR), magnetic bead based assays that utilize fluorescence and chemiluminescence, electrochemiluminescence, mass spectroscopy and detection of enzymatic activity.

Immunoassays can identify and quantify levels of MLDP, aggregates thereof, variants thereof, or fragments thereof. A sample can be contacted with binding entities such as antibodies specific for MLDP and/or related proteins. Complex formation between such binding entities and a particular epitope, or isoform of MLDP, or aggregates thereof, variants thereof, or fragments thereof, can be detected by capture of the complex formed between the binding entity and the MLDP or protein related thereto. In another embodiment, complex formation between binding entities to proteins such as a particular epitope or isoform of MLDP, or aggregates thereof, variants thereof, or fragments thereof, can be detected by use of a detectably labeled secondary antibody. A binding or complex formation event can be recognized and/or quantified by a detectable signal from a reporter molecule. Binding entities such as monoclonal or polyclonal antibodies can be employed.

Such detection methods can include quantifying the amount of expression of MLDP, aggregates thereof, variants thereof, or fragments thereof, for example, by detecting the amount of signal from a labeled MDLP-antibody complex. A variety of immuno-detection methods can be employed for this purpose, including, but not limited to, Western Blot, ELISA, radioimmunoassay, immunocytochemistry, immunohistochemistry, flow cytometry, and immunoprecipitation.

One type of quantitative assay that can be employed is the sandwich or double antibody assay, of which a number of variations exist, all of which are intended to be encompassed by the present invention. For example, in a typical forward sandwich assay, unlabeled antibody is immobilized on a solid substrate, e.g., within microtiter plate wells, and the sample to be tested is brought into contact with the bound antibody. After a suitable period of incubation, for a period of time sufficient to allow formation of an antibody-antigen binary complex, a second antibody, labeled with a reporter molecule capable of emitting or inducing a detectable signal, is then added and incubation is continued allowing sufficient time for binding with the antigen at a different site and the formation of a ternary complex of antibody-antigen-labeled antibody. Any unreacted material is washed away, and the presence of the antigen is determined by observation of a signal from the reporter molecule. The signal can be quantified by comparison with a control sample containing known amounts of antigen (e.g., the Major Lipid Droplet Protein).

Variations on the forward sandwich assay include the simultaneous assay, in which both sample and antibody are added simultaneously to the bound antibody, or a reverse sandwich assay in which the labeled antibody and sample to be tested are first combined, incubated and added to the unlabeled surface bound antibody. These techniques are well known to those skilled in the art, and the possibility of minor variations will be readily apparent. As used herein, “sandwich assay” is intended to encompass all variations on the basic two-site technique.

For the sandwich assays, the binding entities can have different binding specificities or epitope recognition sites. A number of possible combinations are possible. For example, the primary binding entity can bind to MLDP or variant thereof, while a secondary binding entity binds to the primary binding entity. Alternatively, a secondary binding entity can bind to an epitope formed by complex formation between the MLDP (or variant thereof) and the primary binding entity.

By way of example, a device can be obtained or generated in which a primary binding entity is linked to a solid surface. Such a device can be washed in preparation for the test sample. In some instances such washing is not necessary. An aliquot of the test sample can be added to the device. The sample can be incubated in or on the device at 25° C. for a period of time sufficient to allow binding of any MLDP or related proteins present to a primary binding entity. While the sample can be removed and the device washed after this incubation, such washing may not be necessary. A second binding entity can be added to the device and the device can be incubated at 25° C. for an additional period of time sufficient to allow the second binding entity to bind to a complex formed between the primary entity and the MLDP or related protein. The second binding entity can be linked to a reporter molecule that can generate a visible signal. After such a second incubation, the device can be washed again and the signal can be detected or quantified to determine the amount of MDLP, or related protein, in the sample and thereby detect or quantify the oil content of in the algae sample.

If an enzyme-labeled secondary binding entity is employed, it can be added to the first binding entity-antigen complex and allowed to bind to the complex. The excess reagent can be washed away. A solution containing substrate for the enzyme can then be added to the tertiary complex of primary binding entity-antigen-secondary binding entity. The substrate reacts with the enzyme linked to the secondary binding entity. The formation of product by the enzyme provides a qualitative visual signal, which may be further quantified (e.g., spectrophotometrically), to quantify amount of MLDP or related protein in the sample.

Fluorescent reporter molecules, such as fluorescein or rhodamine, can be chemically coupled to binding entities such as antibodies without altering their binding capacity. When activated by illumination with light of a particular wavelength, the fluorophore absorbs the light energy, inducing a state of excitability in the molecule, followed by emission of the light at a characteristic longer wavelength. The emission appears as a characteristic color visually detectable with a light microscope. As in the enzyme immunoassay described above, the fluorescent-labeled secondary binding entity can bind to the primary binding entity-MLDP complex. After washing the unbound reagent, the remaining ternary complex is then exposed to light of the appropriate wavelength. Detection of fluorescence indicates that MLDP is present. The amount of fluorescence detected is directly correlated with the amount of MLDP in the sample.

In another embodiment, the sample can be evaluated in a single site immunoassay wherein protein in the sample is adhered to a solid substrate either covalently or non-covalently. An unlabeled binding entity is brought into contact with the sample bound on the solid substrate. After a suitable period of incubation, for a period of time sufficient to allow formation of a binding entity-antigen binary complex a second binding entity, labeled with a reporter molecule capable of inducing a detectable signal, is then added and incubation is continued allowing sufficient time for the formation of a ternary complex of MLDP-primary binding entity-secondary binding entity. For the single site immunoassay, the secondary binding entity can be a general antibody (i.e., zenogeneic antibody to immunoglobulin, particularly anti-(IgM and IgG) linked to a reporter molecule) that is capable of binding an antibody that is specific for the MLDP protein of interest.

Immunofluorescence and enzyme immunoassay techniques are both very well can be used to detect and quantify expression of MLDP and related proteins. However, other reporter molecules, such as radioisotopes, chemiluminescent or bioluminescent molecules may also be employed. It will be readily apparent to the skilled artisan how to vary the procedure to suit the required use.

Expression of MLDP can therefore be quantified by a variety of methods that involve quantifying a variety of signals from selected reporter molecules. The quantified expression can be observed or calculated in absolute terms, or can be observed or calculated in comparison with a standard (or series of standards) containing a known amounts of antigen (e.g., know amounts of MLDP or related proteins). In one embodiment, the amount or type signal is directly correlated or proportional to concentration of oil in the sample. A calibration chart, or color wheel can be employed to facilitate quantification of the signal. For example, the calibration chart or color wheel can include fixed colors for different concentration ranges so that a user can compare the colorimetric change induced by the secondary antibody to the chart in order to estimate the oil concentration range in the sample.

The detected quantity of Major Lipid Droplet Protein can be compared to a control (known) amount or concentration of Major Lipid Droplet Protein.

### Reporter Molecules

As used herein, a “reporter molecule” is a molecule that provides an analytically detectable signal. Such a reporter molecule facilitates detection of a probe bound to an MLDP nucleic acid or a binding entity bound to an MLDP protein. In some embodiments, detection is quantifiable, to allow determination of the expression level of MLDP nucleic acids and proteins in the sample.

For example, a probe or a binding entity can be detectably labeled, either directly or indirectly with a reporter molecule. Direct reporter molecules include radioisotopes; enzymes having detectable products (e.g., luciferase, beta-galactosidase, and the like); fluorescent labels (e.g., fluorescein isothiocyanate, rhodamine, phycoerythrin, and the like); fluorescence emitting metals, e.g., ¹⁵²Eu, or others of the lanthanide series, attached to a binding entity such as an antibody; chemiluminescent compounds, e.g., luminol, isoluminol, acridinium salts, and the like; bioluminescent compounds, e.g., luciferin, aequorin (green fluorescent protein), and the like. Indirect reporter molecules include entities that pair with another molecule that produces a signal. For example, a primary binding entity or a primary probe can be an indirect label if a secondary binding entity or a secondary probe with an actual reporter molecule is used to bind to and detect a MLDP-binding entity complex or a MLDP mRNA-probe hybrid. Binding pairs such as antigen-antibody, biotin-avidin, and the like can be composed of an indirect label and binding partner with an actual reporter molecule.

Many commonly used reporter molecules are either fluorophores or enzymes. When the reporter molecule is a fluorescent molecule, the signal can be detected by exciting the fluorochrome with the appropriate wavelength of light and detecting the resulting fluorescence as the signal. Fluorescent compounds, such as fluorescein or rhodamine, can be chemically coupled to nucleic acid probes and antibodies without altering their binding capacity. The fluorescence can be detected visually, by means of photographic film, by the use of electronic detectors such as charge coupled devices or photomultipliers and the like. Such fluorescence signals can also be quantified.

When the reporter molecule is an enzyme, the signal can be detected by providing appropriate substrates for the enzyme and detecting the resulting reaction product as a signal. Simple colorimetric labels can be detected by observing the color and/or amount of color associated as a signal from the reporter molecule.

Commonly used enzymes include horseradish peroxidase, glucose oxidase, beta-galactosidase and alkaline phosphatase, among others. The substrates to be used with the specific enzymes are generally chosen for the production, upon hydrolysis by the corresponding enzyme, of a detectable color change. For example, p-nitrophenyl phosphate is suitable for use with alkaline phosphatase conjugates; for peroxidase conjugates, 1,2-phenylenediamine or toluidine are commonly used.

Enzyme reporter molecules can be linked to probes and/or binding entities by available procedures. For example, an enzyme can be conjugated to a binding entity such as an antibody by means of glutaraldehyde or periodate.

### Solid Support

A device for detection and/or quantification of MLDP and variants thereof can include a solid support, a sample addition area and a detection area. Such a solid support can have one or nucleic acid probes or binding entities immobilized thereon.

Any solid support that allows observation of a detectable change, e.g., a visually detectable change, can be employed in the device and methods of the invention. A suitable solid support can include nitrocellulose, transparent solid surfaces (e.g., glass, quartz, plastics and other polymers), opaque solid surface (e.g., white solid surfaces, such as TLC silica plates, filter paper, glass fiber filters, cellulose nitrate membranes, nylon membranes), and conducting solid surfaces (e.g., indium-tin-oxide (ITO), silicon dioxide (SiO2), silicon oxide (SiO), silicon nitride, etc.). The solid support can be any shape or thickness, but generally is flat and thin. The solid supports may be in the form of tubes, beads, discs or microplates, or any other surfaces suitable for detecting mRNA levels or conducting an immunoassay. In one embodiment, the solid support can be a transparent solid support such as glass (e.g., glass slides) or plastic (e.g., wells of microtiter plates).

An embodiment of the present subject matter can include a porous carrier material as a solid support. Other materials suitable for use in the invention are, for example, various kinds of cellulose fiber-containing materials such as filter paper, chromatographic paper, ion exchange paper, a cellulose acetate film, cellulose acetate discs, cellulose thin-layer chromatography discs, as well as films or such materials as starch, as SEPHADEX which is a 3-dimensional network or matrix of dextran chains cross-linked with epichlorhydrin (product of Pharmacia Fine Chemicals, Uppsala, Sweden and Piscatawny, N.J.), films of plastic material such as polyvinylchloride, ceramic material, and of combinations such as polyvinylchloride-silica.

### Production of Binding Entities

To detect, monitor, and detect oil content in algae highly specific and sensitive binding entities against MLDP and related polypeptides can be produced. The binding entities preferentially bind to MLDP and related proteins. The anti-MLDP binding entities can bind to any epitope on the MLDP protein. For example, the anti-MLDP binding entities can bind to any epitope within MLDP polypeptides having any of SEQ ID NO: 2, 4 and 6. For example, the anti-MLDP binding entities preferably bind with specificity to any peptide or protein having at least a portion of the SEQ ID NO: 2, 4, or 6 sequence.

The MLDP epitopes to which the anti-MLDP binding entities can bind can include any MLDP peptide sequence with a segment length, for example, of about 10-20 amino acids. Thus, peptides with MLDP epitopes can be employed for generating anti-MLDP antibodies and/or binding entities from polypeptides having any of SEQ ID NO: 2, 4, and 6 or any variant or analog thereof. Thus, in some embodiments, the MLDP epitope can be a truncated polypeptide, for example, any of SEQ ID NO: 2, 4, and 6 with any number of amino acids removed from the N-terminal and/or C-terminal end. For example, truncated SEQ ID NO: 2, 4, and 6 polypeptides with 1, 2, 3, 4, 5, 6, 7, 8, 9, 10, 11, 12, 13, 14, 15, 16, 17, 18, 19, 20, 21, 22, 23, 24, 25, 30, 40, 50 or 60 amino acid(s) deleted from the N-terminal and/or C-terminal end can be used as epitopes for generating anti-MLDP antibodies and/or binding entities. In other embodiments, the MLDP epitope can be a polypeptide with one or more amino acid substitutions. For example, the MLDP epitope can be a polypeptide with any of the SEQ ID NO:2, 4, and 6 sequences where 1, 2, 3, 4, 5, 6, 7, 8, 9, 10, 11, 12, 13, 14, 15, 16, 17, 18, 19, 20, 21, 22, 23, 24, 25, 30, 40, 50 or 60 amino acid(s) are replaced with another amino acid. In some embodiments, the substituted amino acid(s) have a similar chemical structure or similar chemical properties.

It can be useful to employ a binding entity that binds to a selected lipid droplet-specific factor (e.g., MLDP) with specificity. For example, the binding entity can have an affinity of about 1×10⁷ M⁻¹ to about 1×10¹⁰ M⁻¹, or about 1×10⁸ M⁻¹ to about 1×10⁹ M⁻¹. For example, the affinity of a binding entity can be measured by detecting and quantifying the formation of a binding entity-MLDP complex, such as an antigen-antibody complex [Ag−Ab]. The formation of such an Ag−Ab complex is at equilibrium with its dissociation, and the equilibrium association constant (K_{A}) of the complex can be calculated as follows:

K_{A}=kₐ/k_{d}=[Ag−Ab]/[Ag][Ab]

In some embodiments the binding entity is an antibody. An antibody that is contemplated for use the devices and methods described herein can have variety of structures or forms, including a whole immunoglobulin, an antibody fragment such as Fv, Fab, and similar fragments, a single chain antibody that include the variable domain complementarity determining regions (CDR), and the like forms, all of which fall under the broad term “antibody,” as used herein. Moreover, the binding regions, or CDR, of antibodies can be placed within the backbone of any convenient binding entity polypeptide.

The term “antibody fragment” refers to a portion of a full-length antibody, generally the antigen binding or variable region. Examples of antibody fragments include Fab, Fab′, F(ab′)₂ and Fv fragments. Papain digestion of antibodies produces two identical antigen binding fragments, called Fab fragments, each with a single antigen binding site, and a residual Fc fragment. Fab fragments thus have an intact light chain and a portion of one heavy chain. Pepsin treatment yields an F(ab′)₂ fragment that has two antigen binding fragments that are capable of cross-linking antigen, and a residual fragment that is termed a pFc′ fragment. Fab′ fragments are obtained after reduction of a pepsin digested antibody, and consist of an intact light chain and a portion of the heavy chain. Two Fab′ fragments are obtained per antibody molecule. Fab′ fragments differ from Fab fragments by the addition of a few residues at the carboxyl terminus of the heavy chain CH1 domain including one or more cysteines from the antibody hinge region.

Fv is the minimum antibody fragment that contains a complete antigen recognition and binding site. This region consists of a dimer of one heavy and one light chain variable domain in a tight, non-covalent association (V_{H}-V_{L} dimer). It is in this configuration that the three CDRs of each variable domain interact to define an antigen binding site on the surface of the V_{H}-V_{L} dimer. Collectively, the six CDRs confer antigen binding specificity to the antibody. However, even a single variable domain (or half of an Fv comprising only three CDRs specific for an antigen) has the ability to recognize and bind antigen, although at a lower affinity than the entire binding site. As used herein, “functional fragment” with respect to antibodies, refers to Fv, F(ab) and F(ab′)₂ fragments.

Additional fragments can include diabodies, linear antibodies, single-chain antibody molecules, and multispecific antibodies formed from antibody fragments. Single chain antibodies are genetically engineered molecules containing the variable region of the light chain, the variable region of the heavy chain, linked by a suitable polypeptide linker as a genetically fused single chain molecule. Such single chain antibodies are also referred to as “single-chain Fv” or “sFv” antibody fragments. Generally, the Fv polypeptide further comprises a polypeptide linker between the VH and VL domains that enables the sFv to form the desired structure for antigen binding. For a review of sFv see Pluckthun in The Pharmacology of Monoclonal Antibodies, vol. 113, Rosenburg and Moore eds. Springer-Verlag, N.Y., pp. 269-315 (1994).

The term “diabodies” refers to a small antibody fragments with two antigen-binding sites, where the fragments comprise a heavy chain variable domain (VH) connected to a light chain variable domain (VL) in the same polypeptide chain (VH-VL). By using a linker that is too short to allow pairing between the two domains on the same chain, the domains are forced to pair with the complementary domains of another chain and create two antigen-binding sites. Diabodies are described more fully in, for example, EP 404,097; WO 93/11161, and Hollinger et al., Proc. Natl. Acad Sci. USA 90: 6444-6448 (1993).

Another form of an antibody fragment is a peptide coding for a single complementarity-determining region (CDR). CDR peptides (“minimal recognition units”) are often involved in antigen recognition and binding. CDR peptides can be obtained by cloning or constructing genes encoding the CDR of an antibody of interest. Such genes are prepared, for example, by using the polymerase chain reaction to synthesize the variable region from RNA of antibody-producing cells. See, for example, Larrick, et al., METHODS: A COMPANION TO METHODS IN ENZYMOLOGY, Vol. 2, page 106 (1991).

Antibody fragments are therefore not full-length antibodies. However, such antibody fragments can have similar or improved immunological properties relative to a full-length antibody. Such antibody fragments may be as small as at least about 4 amino acids, 5 amino acids, 6 amino acids, 7 amino acids, 9 amino acids, about 12 amino acids, about 15 amino acids, about 17 amino acids, about 18 amino acids, about 20 amino acids, about 25 amino acids, about 30 amino acids or more.

In general, an antibody fragment or binding entity of the invention can have any upper size limit so long as it is has similar or improved immunological properties relative to an antibody that binds with specificity to a MLDP polypeptide. For example, smaller binding entities and light chain antibody fragments can have less than about 200 amino acids, less than about 175 amino acids, less than about 150 amino acids, or less than about 120 amino acids if the antibody fragment is related to a light chain antibody subunit. Moreover, larger binding entities and heavy chain antibody fragments can have less than about 425 amino acids, less than about 400 amino acids, less than about 375 amino acids, less than about 350 amino acids, less than about 325 amino acids or less than about 300 amino acids if the antibody fragment is related to a heavy chain antibody subunit.

Antibodies directed against MLDP can be made by any available procedure.

Methods for preparing polyclonal antibodies are available to those skilled in the art. See, for example, Green, et al., Production of Polyclonal Antisera, in: IMMUNOCHEMICAL PROTOCOLS (Manson, ed.), pages 1-5 (Humana Press); Coligan, et al., Production of Polyclonal Antisera in Rabbits, Rats Mice and Hamsters, in: CURRENT PROTOCOLS IN IMMUNOLOGY, section 2.4.1 (1992), which are both hereby incorporated by reference in their entireties.

Methods for preparing monoclonal antibodies are likewise available to one of skill in the art. See, for example, Kohler & Milstein, NATURE, 256:495 (1975); Coligan, et al., in: CURRENT PROTOCOLS IN IMMUNOLOGY, sections 2.5.1-2.6.7 (1992); and Harlow, et al., in: ANTIBODIES: A LABORATORY MANUAL, page 726 (Cold Spring Harbor Pub. (1988)), each of which are hereby incorporated by reference.

Monoclonal antibodies can be isolated and purified from hybridoma cultures by a variety of well-established techniques. Such isolation techniques include affinity chromatography with Protein-A Sepharose, size-exclusion chromatography, and ion-exchange chromatography. See, e.g., Coligan, et al., sections 2.7.1-2.7.12 and sections 2.9.1-2.9.3; Barnes, et al., Purification of Immunoglobulin G (IgG), in: Methods in Molecular Biology, Vol. 10, pages 79-104 (Humana Press (1992).

While standardized procedures are available to generate antibodies, the size of antibodies, the multi-stranded structure of antibodies and the complexity of six binding loops present in antibodies may constitute a hurdle to the improvement and the manufacture of large quantities of antibodies, in some instances. Hence, the invention further contemplates using binding entities, which comprise polypeptides that can recognize and bind to a MLDP or related polypeptide.

A number of proteins can serve as protein scaffolds to which binding domains for MLDP or related polypeptides can be attached and thereby form a suitable binding entity. The binding domains bind or interact with MLDP or related polypeptide while the protein scaffold merely holds and stabilizes the binding domains so that they can bind. A number of protein scaffolds can be used. For example, phage capsid proteins can be used. See, review in Clackson & Wells, Trends Biotechnol. 12:173-184 (1994). Phage capsid proteins have been used as scaffolds for displaying random peptide sequences, including bovine pancreatic trypsin inhibitor (Roberts et al., PNAS 89:2429-2433 (1992)), human growth hormone (Lowman et al., Biochemistry 30:10832-10838 (1991)), Venturini et al., Protein Peptide Letters 1:70-75 (1994)), and the IgG binding domain of Streptococcus (O'Neil et al., Techniques in Protein Chemistry V (Crabb, L,. ed.) pp. 517-524, Academic Press, San Diego (1994)). These scaffolds have displayed a single randomized loop or region that can be modified to include binding domains for MLDP or related polypeptides.

Researchers have also used the small 74 amino acid α-amylase inhibitor Tendamistat as a presentation scaffold on the filamentous phage M13. McConnell, S. J., & Hoess, R. H., J. Mol. Biol. 250:460-470 (1995). Tendamistat is a β-sheet protein from Streptomyces tendae. It has a number of features that make it an attractive scaffold for binding peptides, including its small size, stability, and the availability of high resolution NMR and X-ray structural data. The overall topology of Tendamistat is similar to that of an immunoglobulin domain, with two β-sheets connected by a series of loops. In contrast to immunoglobulin domains, the β-sheets of Tendamistat are held together with two rather than one disulfide bond, accounting for the considerable stability of the protein. The loops of Tendamistat can serve a similar function to the CDR loops found in immunoglobulins and can be easily randomized by in vitro mutagenesis. Tendamistat is derived from Streptomyces tendae and may be antigenic in humans. Hence, binding entities that employ Tendamistat are preferably employed in vitro.

Fibronectin type III domain has also been used as a protein scaffold to which binding domains can be incorporated. Fibronectin type III is part of a large subfamily (Fn3 family or s-type Ig family) of the immunoglobulin superfamily. Sequences, vectors and cloning procedures for using such a fibronectin type III domain as a protein scaffold for binding entities (e.g. CDR peptides) are provided, for example, in U.S. Patent Application Publication 20020019517. See also, Bork, P. & Doolittle, R. F. (1992) Proposed acquisition of an animal protein domain by bacteria. Proc. Natl. Acad. Sci. USA 89, 8990-8994; Jones, E. Y. (1993) The immunoglobulin superfamily Curr. Opinion Struct. Biol. 3, 846-852; Bork, P., Hom, L. & Sander, C. (1994) The immunoglobulin fold. Structural classification, sequence patterns and common core. J. Mol. Biol. 242, 309-320; Campbell, I. D. & Spitzfaden, C. (1994) Building proteins with fibronectin type III modules Structure 2, 233-337; Harpez, Y. & Chothia, C. (1994).

Antibodies and binding entities that specifically bind MLDP and related proteins can be used to detect, monitor and quantify oil accumulation in Chlamydomonas reinhardtii and other algae species.

### Kits

Also within the scope of the disclosure are kits that include capture and/or detection binding entities either in the form of a device or as compositions. Instructions for use are also provided in the kits. The kits are useful for detecting the presence of MLDP, aggregates thereof, variants thereof, fragments thereof, or complexes thereof, in a sample. For example, the kit can comprise: one or more capture binding entities (e.g., one or more antibodies) and a detection means for determining the amount of MLDP in the sample. One or more of the capture binding entities may be labeled with a reporter molecule. The kit components, (e.g., reagents) can be packaged in a suitable container. The kit can further comprise instructions for using the kit to detect MLDP.

The kit can also include, e.g., a buffering agent, a preservative or a protein-stabilizing agent. The kit can further include components necessary for performing the detection methods previously discussed. The kit can also contain a control sample or a series of control samples, which can be assayed and compared to the test sample. Such a control can be an known amount of Major Lipid Droplet Protein nucleic acid (DNA or RNA) or Major Lipid Droplet Protein polypeptide. Each component of the kit can be enclosed within an individual container and all of the various containers can be within a single package, along with instructions for interpreting the results of the assays performed using the kit. The kit can contain a written product on or in the kit container. The written product describes how to use the reagents contained in the kit. In several embodiments, the use of the reagents can be according to the methods described herein.

The kit can include a simple colorimetric readout to detect and/or quantify MLDP and thereby detect and/or quantify oil content in a sample.

### DEFINITIONS

As used herein, the term “isolated” refers to a factor (e.g., a polypeptide or nucleic acid) that has been removed from its natural source. For example, an isolated factor can be an in vitro preparation and/or isolation of a nucleic acid molecule, e.g., vector or plasmid, or peptide or polypeptide (protein), or cell, so that it is not associated with in vivo substances. Thus, for example, an “isolated oligonucleotide,” “isolated polynucleotide,” “isolated protein,” or “isolated polypeptide” refers to a nucleic acid or amino acid sequence that is identified and separated from at least one contaminant with which it is ordinarily associated with its source. An isolated nucleic acid or isolated polypeptide can be present in a form or setting that is different from that in which it is found in nature. In contrast, non-isolated nucleic acids (e.g., DNA and RNA) or non-isolated polypeptides (e.g., proteins and enzymes) are found in the state they exist in nature. For example, a given DNA sequence (e.g., a gene) is found on the host cell chromosome in proximity to neighboring genes; RNA sequences (e.g., a specific mRNA sequence encoding a specific protein), are found in the cell as a mixture with numerous other mRNAs that encode a multitude of proteins. However, isolated nucleic acid includes, by way of example, can also be a nucleic acid that is in a chromosomal location different from where it is naturally located, or is otherwise flanked by a different nucleic acid sequence than that found in nature. The isolated nucleic acid or oligonucleotide may be present in single stranded or double stranded form. When an isolated nucleic acid or oligonucleotide is to be utilized to express a protein, the oligonucleotide contains at a minimum, the sense or coding strand (i.e., a single stranded nucleic acid), but may contain both the sense and anti-sense strands (i.e., a double stranded nucleic acid).

The term “nucleic acid molecule,” “polynucleotide,” or “nucleic acid sequence,” as used herein, refers to nucleic acid, DNA or RNA that comprises coding sequences necessary for the production of a polypeptide or protein precursor. The encoded polypeptide may be a full-length polypeptide, a fragment thereof (less than full-length), or a fusion of either the full-length polypeptide or fragment thereof with another polypeptide, yielding a fusion polypeptide.

By “peptide,” “protein,” and “polypeptide” as used herein refers to any chain of amino acids, regardless of length or post-translational modification (e.g., glycosylation or phosphorylation). The nucleic acid molecules of the invention encode a naturally-occurring protein or polypeptide fragment thereof, which has an amino acid sequence that is at least 60%, e.g., at least 65%, 70%, 75%, 80%, 85%, 90%, 95%, 96%, 97%, 98%, 99%, or 100%, amino acid sequence identity to SEQ ID NO: 2, 4, or 6.

The term “homology” as used herein, refers to a degree of complementarity between two or more sequences. There may be partial homology or complete homology (i.e., identity). Homology is often measured using sequence analysis software (e.g., “GCG” and “Seqweb” Sequence Analysis Software Package formerly sold by the Genetics Computer Group, University of Wisconsin Biotechnology Center. 1710 University Avenue, Madison, Wis. 53705). Such software matches similar sequences by assigning degrees of homology to various substitutions, deletions, insertions, and other modifications.

The term “lipid droplet” as used herein, refers to lipid storage organelles or lipid storage depots. Lipid droplets have a structure characterized by a hydrophobic core made up of storage lipids (such as triacylglycerols) surrounded by a phospholipid monolayer to which numerous proteins are attached.

As used herein, the term “antibody” means a polypeptide comprising a framework region from an immunoglobulin gene or fragments thereof that specifically binds and recognizes an antigen. Use of the term antibody is meant to include whole antibodies, including single chain whole antibodies, antibody fragments such as Fab fragments, and other antigen-binding fragments thereof. The term “antibody” includes bispecific antibodies and multispecific antibodies so long as they exhibit the desired biological activity or function.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to conventional polyclonal antibody preparations that typically include different antibodies directed against different determinants (epitopes), each monoclonal antibody is directed against a single determinant on the antigen. The modifier “monoclonal” indicates that the antibody preparation is a substantially homogeneous population of antibodies, and is not to be construed as requiring production of the antibody by any particular method.

As used herein, the term “polyclonal antibody” means a preparation of antibodies derived from at least two (2) different antibody-producing cell lines. The use of this term includes preparations of at least two (2) antibodies that contain antibodies that specifically bind to different epitopes or regions of an antigen.

As used herein, the term “solid support” refers to any surface capable of having capture antibodies bound thereto. Suitable solid support surfaces include, but are not limited to, glass, metal, plastic, or materials coated with a functional group designed for binding of capture antibodies.

As used herein, the term “control” refers to a standard algal or triacylglycerol fatty acid sample with a known amount of triacylglycerol fatty acids; or to an algal sample obtained at an earlier time from the same source as a test sample obtained at a later time. For example, a control can be an aliquot obtained at a selected time and one or more test samples can be obtained at one or more later times, where the amount of triacylglycerol fatty acids in the control can be determined and compared to the one or more test samples that are later obtained.

Unless defined otherwise, all technical and scientific terms used herein generally have the same meaning as commonly understood by one of ordinary skill in the art to which this invention belongs. As used in this specification and the appended claims, the singular forms “a,” “an,” and “the” include plural referents unless the content clearly dictates otherwise. For example, reference to “a cell” includes a combination of two or more cells, and the like. Generally, the nomenclature used herein and the laboratory procedures in cell culture, molecular genetics, organic chemistry, analytical chemistry and nucleic acid chemistry and hybridization described below are those well-known and commonly employed in the art.

The following non-limiting Examples illustrate materials and methods used for development of the subject matter described herein.

### Example 1

### Materials and Methods

Strains and Growth Conditions.

Chlamydomonas reinhardtii strains were grown in Tris-acetate-phosphate (TAP) medium with NH₄Cl at 10 mM, in liquid cultures or on agar-solidified plates under continuous light (70-80 μmol m⁻² s⁻¹) as previously described (Riekhof, 2005). To induce nitrogen limitation, mid-log cells grown in 10 mM TAP were centrifuged for 5 minutes at 5,000×g, washed once in TAP lacking NH₄Cl or any other nitrogen source (TAP-N), and resuspended in TAP-N. Cells were counted with a hematocytometer.

Isolation of Lipid Droplets from Nitrogen-Limited C. reinhardtii.

Cells grown in 500 ml TAP-N medium for 24 h were centrifuged 3,000×g for 5 min and resuspended in 10 ml of isolation buffer (50 mM HEPES, pH 7.5, 5 mM MgCl₂, 5 mM KCl, 0.5 M sucrose, 1 mM phenylmethylsulfonylfluoride, 1 mM 2,2′-dipyridyl, with 1× protease inhibitor cocktail (Roche, Branchburg N.J.)). Cells were disrupted with the aid of a Potter homogenizer, using 20 strokes as well as sonication at the lowest energy setting (3 W output, Misonex Sonicator 3000, Newton, Conn.) for 5 sec. The homogenate was over-layered with buffer 2 (same as isolation buffer above, but lacking 0.5 M sucrose) and centrifuged at 100,000×g for 30 min. Lipid droplets floating on the overlay buffer were removed with a bent Pasteur pipette and diluted 10-fold with washing buffer (same as isolation buffer, but with KCl increased to 150 mM). The suspension was over-layered with buffer 2 and centrifuged as described above. This washing process was repeated three times, and lipid droplets were suspended in buffer 2 and stored at −80° C. Thylakoid membranes were isolated according to known methods (see Chua & Bennoun, Proc. Natl. Acad. Sci. USA 72:2175 (1975)), and enriched eyespots were prepared as previously described (see Schmidt et al., Plant Cell 18:1908 (2006)).

Electron Microscopy.

For electron microscopy, centrifuged cell pellets were processed as described by Harris (THE CHLAMYDOMONAS SOURCEBOOK: A COMPREHENSIVE GUIDE TO BIOLOGY AND LABORATORY USE, Academic Press, San Diego (1989)), and transmission electron micrographs were captured using a JEOLIOO CXII instrument (Japan Electron Optics Laboratories, Tokyo, Japan).

Isolation and Characterization of Lipid Droplet Associated Proteins.

The isolation and characterization of lipid droplets can be performed as described by Moellering & Benning, Eukaryot Cell, 9:97 (2010). Briefly, cells cultured in media with no NH₄ induced C. reinhardtii nitrogen limitation. Lipid droplets were isolated from the nitrogen-limited C. reinhardtii. Lipid droplet proteins were identified by mass spectrometry. Approximately 259 proteins with two or more unique peptides were identified. The identified peptides fell into a broad range of protein functional groups. Approximately 45 proteins of unknown function were also identified.

Based on spectral counts for all of the unknown proteins, the most abundant protein in the lipid droplet fraction based on staining, by a factor of 10, was MLDP. The MLDP nucleotide sequence was determined to be SEQ ID NO: 1.

### Example 2

### MLDP Antibodies

To test the specificity of MLDP antibodies, C. reinhardtii cells were grown in media containing nitrogen (N-replete) and media lacking nitrogen (N-deprived for 48 hours). Cellular extracts and MLDP antiserum were used for immunoblot analysis. Prior to blotting, the MLDP was diluted 1 in 1,000 as a negative control, and the MLDP test-bleed was diluted 1:1,000 and 1:10,000. A single band of about 28 kDa was detected in nitrogen depleted samples with either the 1:1,000 or 1:10,000 dilution of test-bleed, indicating that the MLDP antiserum is specific (FIG. 1).

### Example 3

### The Abundance of MLDP Directly Correlates with Triacylglycerol Levels

The MLDP antiserum was used to test the abundance of MLDP and to correlate MLDP levels with triacylglycerol levels. During nitrogen deprivation, major intracellular changes occur that are likely accompanied by remodeling of membranes (see Miller et al. PLANT PHYSIOLOGY, 154:1737 (2010)). To investigate whether these intracellular changes likely involve MLDP, cells were grown in nitrogen deprived (−N) medium, for 0-60 hours, followed by 0-36 hours of resupplied nitrogen. Samples were taken at various time points and immunoblots were performed. Gas chromatography with flame ionization detectors (GC-FID) was also performed to investigate the levels of triacylglycerol fatty acid to total fatty acid. During nitrogen deprivation, the percentage of triacylglycerol fatty acids to total fatty acids increased as nitrogen deprivation increased, and gradually recovered when nitrogen was resupplied (FIG. 2).

### Example 4

### MLDP is Distributed at the Surface of the Lipid Droplet

Conventional thin-section electron microscopy combined with immunogold labeling was used to visualize MLDP proteins. Prior to visualization, the formation of lipid droplets was induced by nitrogen deprivation. The cell wall-less strain dw15 was deprived of nitrogen for 24 hours. Following nitrogen deprivation, major intracellular changes occur, which are likely accompanied by remodeling of membranes (Miller et al. PLANT PHYSIOLOGY, 154:1737 (2010)). Such membrane remodeling can likely explain the discontinuous feature of plasma membrane and organelle membranes detected by electron microscopy (FIG. 3).

To avoid masking the antigenic sites of MLDP, post-fixation with osmium tetraoxide was avoided, but lipid droplets as electron lucent vesicles were still clearly observed. MLDP was detected with 15 nm electron-dense gold markers localized mainly at the surface of lipid droplets (FIG. 3). Occasionally the gold particles could be detected throughout the lipid droplet core (FIG. 3D), which is very similar to the distribution of perilipin in macrophages and adipocytes (see Robenek et al., J BIOCHEM, 280:26330 (2005)). This localization is most likely explained by differences in sectioning depth and imperfect lipid droplet fusion.

### Example 5

### MLDP can Recruit Different Proteins During Lipogenesis and Lipolysis

In mammalian cells, CGI-58 is in a complex with perilipin under basal conditions and is released to the cytosol upon lipolysis (see Granneman & Moore, TRENDS ENDOCRIN MET, 19:3 (2009)). Experiments were performed to analyze whether MLDP forms a complex in vivo. Lipid droplets were isolated from the nitrogen-limited C. reinhardtii. Proteins from the nitrogen depleted lipid droplets were separated by 4-16% high definition native polyacrylamide gel electrophoresis gel in the first dimension (left to right) and 4-20% sodium dodecyl sulfate polyacrylamide gel electrophoresis in the second dimension (top to bottom) followed by immunoblot with anti-MLDP antibody. Immunoblots of the two dimensional gels were probed with MLDP antiserum that specifically recognized MLDP in a complex between 100 and 140 kDa (FIG. 4A). MLDP-complexes were further compared in nitrogen-depleted (ND) and nitrogen rescued cells (NR) by one-dimensional high definition native polyacrylamide gel electrophoresis (FIG. 4B). The perceived size shift indicates that MLDP may recruit different sets of proteins during lipogenesis versus lipolysis or that MLDP has a different composition of complex components during lipogenesis versus lipolysis.

### Example 6

### Proteins in MLDP-Complexes During Lipogenesis

Based on Coomassie brilliant blue staining, a distinct band was visible by one-dimensional high definition native polyacrylamide gel electrophoresis. Spots representing subunits following analysis by sodium dodecyl sulfate polyacrylamide gel electrophoresis were excised and analyzed by liquid chromatography-tandem mass spectrometry. Forty-three proteins with two or more unique peptides were identified in the MLDP-complex in the state of lipogenesis. Most had previously been associated with lipid droplets (Moellering & Benning, Eukaryot Cell, 9:97 (2010)). The most abundant protein was MLDP, further confirming a role for MLDP as part of a key complex (results not shown).

### Discussion

The foregoing Examples demonstrate a strong correlation between MLDP abundance and the amount of triacylglycerols. To analyze MLDP and its role in lipid droplet dynamics, a MLDP specific antibody was developed. As disclosed in the examples herein, MLDP expression levels directly correlate with the amount of triacylglycerol in algal cells, as shown in nitrogen-deprived cells by immunoblot analysis. As nitrogen was resupplied, MLDP abundance decreased as triacylglycerol decreased. Immunogold-labeling of MLDP shows visual evidence of the subcellular localization of MLDP. Examples disclosed herein provide evidence of major distribution of MLDP at the surface of lipid droplets. Liquid chromatography-tandem mass spectrometry revealed that MLDP was the most abundant protein associated with lipid droplets.

### REFERENCES

Abramoff, M. D., P. J. Magelhaes, and S. J. Ram. 2004. Image processing with ImageJ. Biophotonics Intern. 11:36-42.
Allen, M. D., J. Kropat, S. Tottey, J. A. Del Campo, and S. S. Merchant. 2007. Manganese deficiency in Chlamydomonas results in loss of photo system II and MnSOD function, sensitivity to peroxides, and secondary phosphorus and iron deficiency. Plant Physiol 143:263-277.
Altschul, S. F., T. L. Madden, A. A. Schaffer, J. Zhang, Z. Zhang, W. Miller, and D. J. Lipman. 1997. Gapped BLAST and PSI-BLAST: a new generation of protein database search programs. NucleicAcids Res 25:3389-3402.
Athenstaedt, K., D. Zweytick, A. Jandrositz, S. D. Kohlwein, and G. Daum. 1999. Identification and characterization of major lipid particle proteins of the yeast Saccharomyces cerevisiae. J. Bacteriol. 181:6441-6448.
Atteia, A., A. Adrait, S. Brugiere, M. Tardif, L. R. van, O. Deusch, T. Dagan, L. Kuhn, B. Gontero, W. Martin, J. Garin, J. Joyard, and N. Rolland. 2009. A proteomic survey of Chlamydomonas reinhardtii mitochondria sheds new light on the metabolic plasticity of the organelle and on the nature of the alphaproteobacterial mitochondrial ancestor. Mol. Biol. Evol. 26:1533-1548.
Bartz, R., J. K. Zehmer, M. Zhu, Y. Chen, G. Serrero, Y. Zhao, and P. Liu. 2007. Dynamic activity oflipid droplets: protein phosphorylation and GTPmediated protein translocation. J. Proteome. Res. 6:3256-3265.
Beller, M., C. Sztalryd, N. Southall, M. Bell, H. Jackie, D. S. Auld, and B. Oliver. 2008. COPI complex is a regulator of lipid homeostasis. PLoS. Biol. 6:e292.
Cermelli, S., Y. Guo, S. P. Gross, and M. A. Welte. 2006. The lipid-droplet proteome reveals that droplets are a protein-storage depot. Current Biology 16:1783-1795.
Chua, N. H. and P. Bennoun. 1975. Thylakoid membrane polypeptides of Chlamydomonas reinhardtii: wild-type and mutant strains deficient in photosystem II reaction center. Proc. Natl. Acad. Sci. U.S.A 72:2175-2179.
Elsey, D., D. Jameson, B. Raleigh, and M. J. Cooney. 2007. Fluorescent measurement ofmicroalgal neutral lipids. J. Microbiol. Methods 68:639-642.
Guo, Y., T. C. Walther, M. Rao, N. Stuurman, G. Goshima, K. Terayama, J. S. Wong, R. D. Vale, P. Walter, and R. V. Farese. 2008. Functional genomic screen reveals genes involved in lipid-droplet formation and utilization. Nature 453:657-661.
Harris, E. H.1989. The Chlamydomonas sourcebook: a comprehensive guide to biology and laboratory use. (Academic Press, San Diego).
Hu, Q., M. Sommerfeld, E. Jarvis, M. Ghirardi, M. Posewitz, M. Seibert, and A. Darzins. 2008. Microalgal triacylglycerols as feedstocks for biofuel production: perspectives and advances. Plant Journal 54:621-639.
Huang, A. H. C. 1992. Oil bodies and oleosins in seeds. Annu Rev. Plant Physiol. Plant Mol. Biol. 43:177-200.
Jolivet, P., E. Roux, S. d'Andrea, M. Davanture, L. Negroni, M. Zivy, and T. Chardot. 2004. Protein composition of oil bodies in Arabidopsis thaliana ecotype WS. Plant Physiology and Biochemistry 42:501-509.
Katavic, V., G. K. Agrawal, M. Hajduch, S. L. Harris, and J. J. Thelen. 2006. Protein and lipid composition analysis of oil bodies from two Brassica napus cultivars. Proteomics. 6:4586-4598.
Kyte, J. and R. F. Doolittle. 1982. A simple method for displaying the hydropathic character of a protein. J. Mol. Biol. 157:105-132.
Larionov, A., A. Krause, and W. Miller. 2005. A standard curve based method for relative real time PCR data processing. BMC. Bioinformatics. 6:62.
Londos, C., C. Sztalryd, J. T. Tansey, and A. R. Kimmel. 2005. Role of PAT proteins in lipid metabolism. Biochimie 87:45-49.
Lumbreras, V., D. R. Stevens, and S. Purton. 1998. Efficient foreign gene expression in Chlamydomonas reinhardtii mediated by an endogenous intron. Plant J. 14:441-447.
Merchant, S. S., S. E. Prochnik, O. Vallon, E. H. Harris, S. J. Karpowicz, G. B. Witman, A. Terry, A. Salamov, L. K. Fritz-Laylin, L. Marechal-Drouard, W. F. Marshall, L. H. Qu, D. R. Nelson, A. A. Sanderfoot, M. H. Spalding, V. V. Kapitonov, Q. Ren, P. Ferris, E. Lindquist, H. Shapiro, S. M. Lucas, J. Grimwood, J. Schmutz, P. Cardol, H. Cerutti, G. Chanfreau, C. L. Chen, V. Cognat, M. T. Croft, R. Dent, S. Dutcher, E. Fernandez, H. Fukuzawa, D. Gonzalez-Ballester, D. Gonzalez-Halphen, A. Hallmann, M. Hanikenne, M. Hippler, W. Inwood, K. Jabbari, M. Kalanon, R. Kuras, P. A. Lefebvre, S. D. Lemaire, A. V. Lobanov, M. Lohr, A. Manuell, I. Meier, L. Mets, M. Mittag, T. Mittelmeier, J. V. Moroney, J. Moseley, C. Napoli, A. M. Nedelcu, K. Niyogi, S. V. Novoselov, I. T. Paulsen, G. Pazour, S. Purton, J. P. Ral, D. M. RianoPachon, W. Riekhof, L. Rymarquis, M. Schroda, D. Stem, J. Umen, R. Willows, N. Wilson, S. L. Zimmer, J. Allmer, J. Balk, K. Bisova, C. J. Chen, M. Elias, K. Gendler, C. Hauser, M. R. Lamb, H. Ledford, J. C. Long, J. Minagawa, M. D. Page, J. Pan, W. Pootakham, S. Roje, A. Rose, E. Stahlberg, A. M. Teraucbi, P. Yang, S. Ball, C. Bowler, C. L. Dieckmann, V. N. Gladysbev, P. Green, R. Jorgensen, S. Mayfield, B. Mueller-Roeber, S. Rajamani, R. T. Sayre, P. Brokstein, I. Dubchak, D. Goodstein, L. Hornick, Y. W. Huang, J. Jhaveri, Y. Luo, D. Martinez, W. C. Ngau, B. Otillar, A. Poliakov, A. Porter, L. Szajkowski, G. Werner, K. Zhou, I. V. Grigoriev, D. S. Rokhsar, and A. R. Grossman. 2007. The Chlamydomonas genome reveals the evolution of key animal and plant functions. Science 318:245-250.
Moellering, E. R. and Benning C. 2010 RNA interference silencing of a major lipid droplet affects lipid droplet size in Chlamydomonas reinhardtii. Eukaryot Cell. 9:97-106.
Murphy, D. J. 2001. The biogenesis and functions of lipid bodies in animals, plants and microorganisms. Prog. Lipid Res. 40:325-438.
Nesvizhskii, A. I., A. Keller, E. Kolker, and R. Aebersold. 2003. A statistical model for identifying proteins by tandem mass spectrometry. Anal. Chem. 75:4646-4658.
Pazour, G. J., N. Agrin, J. Leszyk, and G. B. Witman. 2005. Proteomic analysis of a eukaryotic cilium. J. Cell Biol. 170:103-113.
Riekhof, W. R., B. B. Sears, and C. Benning 2005 Annotation of genes involved in glycerolipid biosynthesis in Chlamydomonas reinhardtii: discovery of the betaine lipid synthase BTAlCr. Eukaryot. Cell 4:242-252.
Rossak, M., A. Schafer, N. Xu, D. A. Gage, and C. Benning 1997. Accumulation of sulfoquinovosyl-1-O-dihydroxyacetone in a sulfolipid-deficient mutant of Rhodobacter sphaeroides inactivated in sqdC. Arch. Biochem. Biophys. 340:219-230.
Sato, N. and N. Murata. 1991. Transition of lipid phase in aqueous dispersions of diacylglyceryltrimethylhomoserine. Biochim. Biophys. Acta 1082:108-111.
Schmidt, M., G. Gessner, M. Luff, I. Heiland, V. Wagner, M. Kaminski, S. Geimer, N. Eitzinger, T. Reissenweber, O. Voytsekh, M. Fiedler, M. Mittag, and G. Kreimer. 2006. Proteomic analysis of the eyespot of Chlamydomonas reinhardtii provides novel insights into its components and tactic movements. Plant Cell 18: 1908-1930.
Shevchenko, A., M. Wilm, O. Vorm, and M. Mann. 1996. Mass spectrometric sequencing of proteins silver-stained polyacrylamide gels. Anal. Chem. 68:850-858.
Shimada, T. L., T. Shimada, H. Takahashi, Y. Fukao, and I. Hara-Nishimura. 2008. A novel role for oleosins in freezing tolerance of oilseeds in Arabidopsis thaliana. Plant J. 55:798-809.
Siloto, R. M., K. Findlay, A. Lopez-Villalobos, E. C. Yeung, c. L. Nykiforuk, and M. M. Moloney. 2006. The accumulation of oleosins determines the size of seed oil bodies in Arabidopsis. Plant Cell 18:1961-1974.
Tansey, J. T., C. Sztalryd, J. Gruia-Gray, D. L. Roush, J. V. Zee, O. Gavrilova, M. L. Reitman, C. X. Deng, C. Li, A. R. Kimmel, and C. Londos. 2001. Perilipin ablation results in a lean mouse with aberrant adipocyte lipolysis, enhanced leptin production, and resistance to diet-induced obesity. Proc. Natl. Acad. Sci. U.S.A 98:6494-6499.
Thompson, G. A. 1996. Lipids and membrane function in green algae. Biochimica et Biophysica Acta-Lipids and Lipid Metabolism 1302:17-45.
Van Donk, E., M. Lurling, D. O. Hessen, and G. M. Lokhorst. 1997. Altered cell wall morphology in nutrient-deficient phytoplankton and its impact on grazers. Limnology and Oceanography 42:357-364.
Walther, T. C. and R. V. Farese, Jr. 2009. The life of lipid droplets. Biochim. Biophys. Acta 1791:459-466.

All patents and publications referenced or mentioned herein are indicative of the levels of skill of those skilled in the art to which the invention pertains, and each such referenced patent or publication is hereby specifically incorporated by reference to the same extent as if it had been incorporated by reference in its entirety individually or set forth herein in its entirety. Applicants reserve the right to physically incorporate into this specification any and all materials and information from any such cited patents or publications.

The specific methods and compositions described herein are representative of preferred embodiments and are exemplary and not intended as limitations on the scope of the invention. Other objects, aspects, and embodiments will occur to those skilled in the art upon consideration of this specification, and are encompassed within the spirit of the invention as defined by the scope of the claims. It will be readily apparent to one skilled in the art that varying substitutions and modifications may be made to the invention disclosed herein without departing from the scope and spirit of the invention. The invention illustratively described herein suitably may be practiced in the absence of any element or elements, or limitation or limitations, which is not specifically disclosed herein as essential. The methods and processes illustratively described herein suitably may be practiced in differing orders of steps, and the methods and processes are not necessarily restricted to the orders of steps indicated herein or in the claims.

As used herein and in the appended claims, the singular forms “a,” “an,” and “the” include plural reference unless the context clearly dictates otherwise. Thus, for example, a reference to “a nucleic acid” or “a polypeptide” includes a plurality of such nucleic acids or polypeptides (for example, a solution of nucleic acids or polypeptides or a series of nucleic acid or polypeptide preparations), and so forth. Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims and statements of the invention.

The following statements of the invention are intended to describe and summarize various embodiments of the invention according to the foregoing description in the specification.

### Statements:

1. A method of detecting oil content in an algae sample comprising detecting Major Lipid Droplet Protein (MLDP) nucleic acid content or Major Lipid Droplet Protein (MLDP) protein content to thereby detect oil content in the algae sample.
2. The method of statement 1, wherein the MLDP protein has at least 85%, or at least 90%, or at least 95% amino acid sequence identity to a polypeptide having SEQ ID NO: 2, 4, 6 or 8.
3. The method of statement 1 or 2, wherein the MLDP nucleic acid encodes a polypeptide with at least 85%, or at least 90%, or at least 95% amino acid sequence identity to a polypeptide having SEQ ID NO: 2, 4, 6, or 8.
4. The method of any of statements 1-3, wherein the MLDP nucleic acid has at least 85%, or at least 90%, or at least 95% nucleotide sequence identity to a nucleic acid segment of SEQ ID NO: 1, 3, 5, or 7.
5. The method of any of statements 1-4, wherein the algae sample is a Chlamydomonas, Chlorophyta (green algae), Rhodophyta (red algae), or Phaeophyceae (brown algae) sample.
6. The method of any of statements 1-5, wherein the algae sample is a Chlorophyta (green algae) sample.
7. The method of any of statements 1-6, wherein the algae sample is a Chlamydomonas reinhardtii sample.
8. The method of any of statements 1-7, further comprising obtaining a sample.
9. The method of any of statements 1-8, further comprising obtaining an algae sample from a field, meadow, forest, jungle, swamp, estuary, beach, pond, stream, river, cell culture, fermenter, or bioreactor.
10. The method of any of statements 1-9, wherein detection of a high level of MLDP means the sample has a high level of triacylglycerol.
11. The method of any of statements 1-10, wherein detection of a low level of MLDP means the sample has a low level of triacylglycerol.
12. The method of any of statements 1-11, further comprising quantifying Major Lipid Droplet Protein (MLDP) nucleic acid content or Major Lipid Droplet Protein (MLDP) protein content to quantify oil content in the sample.
13. The method of any of statements 1-12, wherein detecting or quantifying MLDP nucleic acid content comprises microarray analysis, Northern blotting, nuclease protection assays, RNA fingerprinting, polymerase chain reaction, ligase chain reaction, Qbeta replicase, isothermal amplification, strand displacement amplification, transcription based amplification, quantitative nucleic acid amplification assays, polymerase chain reaction assays, combined reverse transcription/nucleic acid amplification, nuclease protection assays, SI nuclease assays, RNAse protection assays, Serial Analysis Gene Expression (SAGE), next generation sequencing, gene expression microarray, or a combination thereof.
14. The method of any of statements 1-13, wherein detecting or quantifying MLDP protein content comprises Western blotting, enzyme-linked immunosorbent assay (ELISA), radioimmunoassay, immunocytochemistry, immunohistochemistry, flow cytometry, immunoprecipitation, electrophoresis, immunoPCR (iPCR), magnetic bead assay, mass spectroscopy or a combination thereof.
15. The method of any of statements 1-14, further comprising monitoring MLDP expression in an algal source over time by obtaining samples from the algal source over time and detecting and/or quantifying MLDP nucleic acids or proteins in the separate samples obtained.
16. The method of any of statements 1-15, wherein detecting or quantifying MLDP protein content comprises an immunoassay.
17. The method of any of statements 1-16, wherein an amount of MLDP in the sample is directly correlated with the oil content of the sample.
18. The method of any of statements 1-17, wherein an amount of MLDP in the sample is directly correlated with the percent triacylglycerol fatty acids in the sample.
19. The method of any of statements 1-18, wherein a 10% increase in MLDP relative to a control, directly relates to a 5-20% increase in the percentage of triacylglycerol fatty acids in the sample relative to the control.
20. The method of any of statements 1-19, wherein when MLDP expression is ten-fold greater than a control, the percentage of triacylglycerol fatty acids relative to total fatty acids in the sample is about 10 to about 20 fold greater than in the control.
21. The method of any of statements 1-20, wherein the Major Lipid Droplet Protein (MLDP) nucleic acid is an mRNA.
22. A method comprising:

contacting a test sample with antibodies specific for polypeptide having at least 90% amino acid sequence identity to a polypeptide having SEQ ID NO: 2, 4 or 6;

incubating the test sample with the antibodies for a time sufficient and under conditions that permit antigen-antibody complex formation;

detecting the presence of said antigen-antibody complex to thereby detect the test sample's oil content.

23. The method of statement 22, wherein detecting the presence of said antigen-antibody complex comprises detecting a reporter molecule covalently or non-covalently bound to the complex.
24. The method of statement 22 or 23, wherein detecting the presence of said antigen-antibody complex comprises contacting the complex with a secondary antibody linked to a reporter molecule.
25. The method of any of statements 22-24, wherein detecting the presence of said antigen-antibody complex comprises contacting the complex with a substrate for an enzyme reporter molecule.
26. The method of statement 25, wherein the enzyme reporter molecule converts the substrate to a detectable signal.
27. The method of any of statements 1-26, wherein detecting or quantifying MLDP comprises detecting or quantifying a colorimetric signal, radioactive signal, electrochemical signal, fluorescent signal or a combination thereof.
28. An apparatus or device comprising:

a solid support comprising antibodies bound thereto;

wherein the antibodies are specific for an polypeptide having at least 70%, or at least 75%, or at least 80%, or at least 85%, or at least 90% amino acid sequence identity to a polypeptide having SEQ ID NO: 2, 4 or 6.

29. An apparatus or device comprising:

a solid support comprising at least one nucleic acid probe bound thereto;

the nucleic acid probe is specific for a mRNA having at least 70%, or at least 75%, or at least 80%, or at least 85%, or at least 90% sequence identity to a nucleic acid having SEQ ID NO: 1, 3, or 5.

30. The apparatus of statement 28 or 29, wherein the solid support comprises nitrocellulose, dextran, glass, quartz, plastic, silica, cellulose nitrate, nylon, polyvinylchloride, polyvinylchloride-silica, indium-tin-oxide (ITO), silicon dioxide (SiO2), silicon oxide (SiO), silicon nitride, gold, steel, iron, aluminum, or a combination thereof.
31. The apparatus of any of statements 28-30, wherein the solid support comprises one or more porous carrier materials.
32. The apparatus of any of statements 28-31, wherein the solid support comprises one or more tubes, beads, discs or microplates.
33. The apparatus of any of statements 28-32, wherein the apparatus is a dipstick.
34. The apparatus of any of statements 28-33, wherein the apparatus is a dipstick comprising a reporter molecule activated by binding of an MLDP nucleic acid or protein to a probe or binding entity specific for the MLDP nucleic acid or protein.
35. The apparatus of any of statements 28-34, configured to emit a signal proportional to amounts of MLDP nucleic acid or an MLDP protein bound.
36. The apparatus of any of statements 28-35, configured to emit a signal identifying when a desired or selected amount of MLDP nucleic acid or MLDP protein is bound.
37. The apparatus of any of statements 28-36, configured to emit a colorimetric signal, radioactive signal, electrochemical signal, fluorescent signal or a combination thereof.
38. An antibody that binds specifically to a polypeptide with a sequence that has at least 95% sequence identity to any of SEQ ID NO: 2, 4, 6 or 8.
39. A kit comprising:

the apparatus of any of statements 27-37; and

instructions for using the apparatus to detect or quantify MLDP and thereby detect or quantify oil content in a sample.

40. The kit of statement 39, wherein the apparatus comprises an antibody that binds specifically to a polypeptide with a sequence that has at least 95% sequence identity to any of SEQ ID NO: 2, 4, 6 or 8.
41. The kit of statement 39 or 40, wherein the instructions correlate the quantity of MLDP with the quantity of oil in the sample.
42. The kit of any of statements 39-41, wherein the instructions inform the user to contact the apparatus with the sample, incubate the sample with the apparatus for a time sufficient to permit nucleic acid probe-mRNA binding or antigen-antibody binding to occur, and/or wash the device before detecting said binding.

The following claims describe aspects of the invention.

What is claimed:

1. A method of quantifying oil content in an algae sample comprising quantifying Major Lipid Droplet Protein (MLDP) nucleic acid content or Major Lipid Droplet Protein (MLDP) protein content to thereby quantify oil content in the algae sample.

2. The method of claim 1, wherein the MLDP protein(s) have at least 75% amino acid sequence identity to a polypeptide having SEQ ID NO: 2, 4, 6, or 8.

3. The method of claim 1, wherein the MLDP nucleic acid(s) encode a polypeptide with at least 75% amino acid sequence identity to a polypeptide having SEQ ID NO: 2, 4, 6, or 8.

4. The method of claim 1, wherein the MLDP nucleic acid(s) have at least 75% nucleotide sequence identity to a nucleic acid segment of SEQ ID NO: 1, 3, 5, or 7.

5. The method of claim 1, wherein the algae sample is a Chlorophyta (green algae) sample.

6. The method of claim 1, further comprising obtaining an algae sample from a field, meadow, forest, jungle, swamp, estuary, beach, pond, stream, river, cell culture, fermenter, or bioreactor.

7. The method of claim 1, wherein quantifying MLDP nucleic acid(s) comprises microarray analysis, Northern blotting, nuclease protection assays, RNA fingerprinting, polymerase chain reaction, ligase chain reaction, Qbeta replicase, isothermal amplification, strand displacement amplification, transcription based amplification, quantitative nucleic acid amplification assays, polymerase chain reaction assays, combined reverse transcription/nucleic acid amplification, nuclease protection assays, SI nuclease assays, RNAse protection assays, Serial Analysis Gene Expression (SAGE), next generation sequencing, gene expression microarray, or a combination thereof.

8. The method of claim 1, wherein quantifying MLDP protein(s) comprises Western blotting, enzyme-linked immunosorbent assay (ELISA), radioimmunoassay, immunocytochemistry, immunohistochemistry, flow cytometry, immunoprecipitation, electrophoresis, immunoPCR (iPCR), magnetic bead assay, mass spectroscopy or a combination thereof.

9. The method of claim 1, wherein quantifying MLDP protein(s) comprises an immunoassay.

10. The method of claim 1, wherein the amount of MLDP in the sample is directly correlated with the oil content.

11. The method of claim 1, further comprising monitoring MLDP expression in a algal source over time by obtaining samples from the algal source over time and separately detecting and/or quantifying MLDP nucleic acids or proteins the obtained samples.

12. The method of claim 1, wherein a 10% increase in MLDP relative to a control, directly relates to a 5-20% increase in the percentage of triacylglycerol fatty acids in the sample relative to the control.

13. The method of claim 1, wherein when MLDP expression is ten-fold greater than a control, the percentage of triacylglycerol fatty acids relative to total fatty acids in the sample is about 10 to about 20 fold greater than in the control.

14. A method comprising:
contacting a sample with antibodies specific for polypeptide having at least 90% amino acid sequence identity to a polypeptide having SEQ ID NO: 2, 4 or 6;
incubating the test sample with the antibodies for a time sufficient and under conditions that permit antigen-antibody complex formation;
detecting the presence of said antigen-antibody complex to thereby detect the sample's oil content.

15. An apparatus or device comprising:
a solid support comprising antibodies bound thereto;
wherein the antibodies are specific for an polypeptide having at least 70%, amino acid sequence identity to a polypeptide having SEQ ID NO: 2, 4 or 6.

16. The apparatus or device of claim 15, wherein the apparatus is a dipstick.

17. The apparatus of claim 15, configured to emit a signal proportional to an amount of MLDP protein bound thereto.

18. The apparatus of claim 15, configured to emit a signal identifying when a desired or selected amount of MLDP protein is bound.

19. An apparatus or device comprising:
a solid support comprising at least one nucleic acid probe bound thereto;
the nucleic acid probe is specific for a mRNA having at least 70% sequence identity to a nucleic acid having SEQ ID NO: 1, 3, or 5.

20. A kit comprising:
(a) a solid support comprising antibodies bound thereto, wherein the antibodies are specific for an polypeptide having at least 70%, amino acid sequence identity to a polypeptide having SEQ ID NO: 2, 4 or 6;
(b) a solid support comprising at least one nucleic acid probe bound thereto;
the nucleic acid probe is specific for a mRNA having at least 70% sequence identity to a nucleic acid having SEQ ID NO: 1, 3, or 5;
(c) or both; and
instructions for using the apparatus to detect or quantify MLDP and thereby detect or quantify oil content in a sample.

21. The kit of claim 20, wherein the instructions correlate the quantity of MLDP with the quantity of oil in the sample.

22. The kit of claim 21, wherein the instructions inform the user to contact the apparatus with the sample, incubate the sample with the apparatus for a time sufficient to permit nucleic acid probe-mRNA binding or antigen-antibody binding to occur, and/or wash the device before detecting said binding.
